(19) United States
(12) Patent Application Publication
(10) Pub. No.: US 2025/0090555 A1
(43) Pub. Date: Mar. 20, 2025

(54) METHOD FOR PRODUCING A CBD COMPRISING LIQUID OR SOLID AND USE THEREOF

(71) Applicant: Food4Innovations Ventures B.V., Wageningen (NL)

(72) Inventors: Jolieke Monica Johanna WILLEMS, Wageningen (NL); Karen Elisabeth BRINK, Wageningen (NL); Wouter Bernardus Cornelius DE HEIJ, Wageningen (NL)

(21) Appl. No.: 18/728,964

(22) PCT Filed: Jan. 13, 2023
(86) PCT No.: PCT/NL2023/050011

§ 371 Date: Jul. 15, 2024

(30) Foreign Application Priority Data
Jan. 14, 2022 (NL) 2030552
Feb. 4, 2022 (NL) 2030822

(51) Int. Cl. A61K 31/00; A61K 47/12; A61K 47/14; A61K 47/22; A61K 47/24; A61K 47/26; A61K 47/36; A61K 47/42; A61K 47/44; A61K 47/46

(52) U.S. Cl. CPC A61K 31/658; A61K 47/12; A61K 47/14; A61K 47/22; A61K 47/24; A61K 47/26; A61K 47/36; A61K 47/42; A61K 47/44; A61K 47/46

(57) ABSTRACT

The invention provides a method for producing a liquid, wherein the method comprises a first stage for providing a first liquid comprising components, wherein the components comprise water, oil, an emulsifier, and a functional component, wherein the functional component comprises Cannabidiol, wherein the first stage comprises: (a) a first premixing stage, wherein the first premixing stage comprises: combining and mixing the Cannabidiol, oil, first emulsifier, and optionally a further first premixing stage mixture component, to provide a first premixing stage mixture; (b) a first mixing stage, wherein the first mixing stage comprises: combining and mixing water and the first premixing stage mixture to provide a first mixture, wherein in the first mixing stage a mixer device is applied rotating at a speed of at least 5000 RPM; and wherein the functional component comprises Cannabidiol; and (c) a first homogenization stage to provide the first liquid, wherein the first homogenization stage comprises homogenizing in a homogenizer n times the first mixture, wherein in the first homogenization stage a homogenizer is applied at a pressure of at least 250 bar; wherein n is selected from the range of 1-10.

### FIELD OF THE INVENTION

The invention relates to a method for producing a liquid, the liquid as such, as well as the use thereof. The invention also relates to a method for producing a solid, and optionally a liquid thereof, as well as its use. The invention also relates to the products obtainable with the herein described method(s).

### BACKGROUND OF THE INVENTION

The use of cannabinoid is known in the art. EP3782602, for instance, describes a composition comprising surfactant-enhanced phospholipid vesicles with one or more cannabinoid substance encapsulated therein, wherein one or more surfactant is utilized for enhancing loading and increasing encapsulation efficiency of cannabinoid passenger molecules within phospholipid structures. A method is described for making a surfactant-enhanced phospholipid vesicles with one or more cannabinoid substance encapsulated therein, wherein one or more surfactant is used for enhancing loading and increasing encapsulation efficiency of passenger molecules in phospholipid structures. A method of using surfactant-enhanced phospholipid vesicles with one or more cannabinoid substance encapsulated therein is described wherein one or more surfactants enhance loading and increases encapsulation efficiency of cannabinoid substances in phospholipid structures. A composition and method of making surfactant-enhanced phospholipid vesicles with one or more lipophilic passenger substance encapsulated therein is described wherein one or more surfactant is utilized for enhancing loading and increasing encapsulation efficiency of passenger molecules within phospholipid structures.

N. M. Francke, F. Schneider, K. Baumann, H. Bunjes, Formulation of Cannabidiol in Colloidal Lipid Carriers, Molecules. 26 (2021) 1469. https://doi.org/10.3390/molecules26051469, describes an investigation into the general processability of cannabidiol (CBD) in colloidal lipid carriers.

US2019015383A1, describes water-soluble cannabinoid compositions. The composition may include a cannabinoid mixture, e.g., purified from a cannabis extract, and one or more water soluble agents, such as a complex carbohydrate. Methods of preparing such compositions may include blending the cannabinoid mixture with the water soluble agents(s) in water to form an emulsion. Some methods include drying the emulsion to form a film coating or particles, such as spray-dried and/or agglomerated particles, wherein the film coating or particles are at least partially soluble in cold water.

N. M. Francke, L. Grüne, H. Bunjes, Formulation of Cannabidiol in Lipid Carriers, SPhERe Proceedings (2020). https://doi.org/10.24355/dbbs.084-202001221033-0, describes an investigation into different types of lipid carriers as formulation options for cannabidiol to enable parenteral or oral application.

### SUMMARY OF THE INVENTION

There appears to be a desire to provide a cannabinoid substance in an easily useable form, and preferably with an enhanced uptake. Solutions known in the art may be less desirable in terms of taste, complexity and/or uptake by the human body.

Hence, amongst others it is an aspect of the invention to provide an alternative cannabinoid comprising material and/or method for the production thereof, which preferably further at least partly obviates one or more of above-described drawbacks. The present invention may have as object to overcome or ameliorate at least one of the disadvantages of the prior art, or to provide a useful alternative.

According to a first aspect, the invention provides a method for producing a liquid. Especially, the method comprises a first stage for providing a first liquid comprising components. The components may comprise water, oil, an emulsifier, and a functional component. In embodiments, the first stage may comprise a first mixing stage and a first homogenization stage. In embodiments, the first mixing stage may comprise: combining and mixing water, oil, an emulsifier, and the functional component, to provide a first mixture. Especially, in embodiments in the first mixing stage a mixer device, such as especially an ultra turrax, is applied rotating at a speed of at least 5000 RPM. Further, in embodiments the functional component comprises Cannabidiol (CBD). Further, in embodiments in the first homogenization stage the first liquid is provided. Especially, in embodiments the first homogenization stage comprises homogenizing in a homogenizer n times the first mixture. Especially, in embodiments in the first homogenization stage a homogenizer is applied at a pressure of at least 250 bar. Further, in embodiments n may be selected from the range of 1-10. Hence, especially in embodiments the invention provides a method for producing a liquid, wherein the method comprises a first stage for providing a first liquid comprising components, wherein the components comprise water, oil, an emulsifier, and a functional component, wherein the first stage comprises: (A) a first mixing stage, wherein the first mixing stage comprises: combining and mixing water, oil, an emulsifier, and the functional component, to provide a first mixture, wherein in the first mixing stage a mixer device is applied; and wherein the functional component comprises Cannabidiol; and (B) a first homogenization stage to provide the first liquid, wherein the first homogenization stage comprises homogenizing in a homogenizer n times the first mixture, wherein in the first homogenization stage a homogenizer is applied at a pressure of at least 250 bar; wherein n is selected from the range of 1-10. Especially, in embodiments in the first mixing stage a mixer device is applied rotating at a speed of at least 5000 RPM, more especially wherein the mixer device is an ultra turrax device.

Hence, the phrase “the first homogenization stage comprises homogenizing in a homogenizer n times the first mixture”, and similar phrases, may indicate that the first homogenization stage may comprise n homogenization actions or n homogenization substages, which are herein further also (simply) indicated as “n homogenizations”, and similar indications.

With such a method a cannabinoid substance may be provided in an easily useable form, such as after dilution. Further, with such a method, a cannabinoid substance may be provided having an enhanced uptake (in the human body). Further, such cannabinoid substance may be more desirable in terms of taste compared to known solutions. Further, the method may be relatively simple. Further, when diluting the cannabinoid, a clear solution may be provided, in embodiments, comparable to water, while nevertheless comprising the cannabinoid substance in a desirable concentration.

As indicated above, the invention provides a method for producing a liquid.

The method especially comprises a first stage, leading to a first liquid, and optionally a second stage, leading to a second liquid. The first liquid may also be concentrate to a solid material, which may optionally be diluted again to obtain the second liquid. Hence, the term “liquid” in the phrase “a method for producing a liquid”, and similar phrases, may refer in embodiments to the first liquid and may in (other) embodiments refer to the second liquid. Here below, the invention is especially described in relation to the first liquid and the second liquid, respectively.

Especially, the method may comprise a first stage for providing a first liquid comprising components. The first liquid may not be clear, and may have a kind of milky appearance. After dilution, however, the liquid (then indicated as second liquid, see also below), may be clear.

In embodiments, the components comprise water, oil, an emulsifier, and a functional component. The term “oil” may refer to one or more different oils. Especially, the oils are edible oils. The term “emulsifier” may refer to one or more different emulsifiers. Especially, the emulsifiers are edible emulsifiers. The term “functional component” may refer to one or more different functional components. Especially, the functional components are edible functional components. The listing of the components water, oil, an emulsifier, and a functional component does not imply that further components may not be available. In embodiments, one or more further components may be available (such as e.g. first premixing stage mixture component, see also below).

In embodiments, the first stage may comprise a first mixing stage. Especially, the first mixing stage may comprise: combining and mixing water, the oil, the emulsifier, and the functional component, to provide a first mixture. Especially, in embodiments in the first mixing stage a mixer device may be applied rotating at a speed of at least 5000 RPM.

Especially, in embodiments the functional component may comprise Cannabidiol. Cannabidiol is also known as CBD and is considered a phytocannabinoid. CBD may have a positive impact on one or more of anxiety, cognition, movement disorders, and pain. CBD may also have a positive impact on epilepsy disorders.

In embodiments, the first stage may comprise a first homogenization stage to provide the first liquid. Especially, the first homogenization stage may comprise homogenizing in a homogenizer n times the first mixture. In the first homogenization stage a homogenizer may be applied at a pressure of at least 250 bar. Especially, n may be selected from the range of 1-10.

Especially, the first homogenization stage is subsequent to the first mixing stage, though there may be some time between the mixing stage and the subsequent homogenization stage (see also below).

Especially, (i) the components may be selected such and (ii) the first stage may be executed such that the first liquid has a turbidity of not more than 1000 Nephelometric Turbidity units. Nephelometric Turbidity units are also known as NTUs.

The NTU value may be determined by a nephelometer. Nephelometers are known in the art. As indicated by Wikipedia, a nephelometer or aerosol photometer is an instrument for measuring the concentration of suspended particulates in a liquid or gas colloid. A nephelometer measures suspended particulates by employing a light beam (source beam) and a light detector set to one side (often) 90° of the source beam.

More especially, (i) the components may be selected such and (ii) the first stage may be executed such that the first liquid has a turbidity of not more than 500 Nephelometric Turbidity units.

The term “emulsifier” may in embodiments refer to a first emulsifier and a second emulsifier. Hence, the term “emulsifier” may refer to one or more emulsifier. Hence, in embodiments two or more different emulsifiers may be applied.

The term “oil” may especially refer to a food-grade oil. The term “oil” may refer to one or more oils. Hence, in embodiment two or more different oils may be applied.

In embodiments, the components comprise a first emulsifier.

Especially, in embodiments the first mixing stage may comprise a first premixing stage. The first premixing stage may comprise: (i) combining and mixing the Cannabidiol, a (food-grade) oil, the first emulsifier, and optionally a further first premixing stage mixture component, and optionally (ii) subjecting the thus obtained first premixing stage mixture to a heating stage. Especially, in embodiments the thus obtained mixture (“first premixing stage mixture” or “Cannabidiol comprising premixing stage mixture”) may be subjected to a temperature selected from the range of 20-60° C. More especially, the thus obtained mixture may be subjected to a temperature selected from the range of 30-60° C. Especially, the thus obtained mixture may be subjected to such temperature during a time period of at least 15 minutes. For instance, the time period may be selected from the range of 15-60 minutes. However, other time periods for subjecting the thus obtained mixture to a temperature higher than room temperature (i.e. higher than 20° C.) may also be possible.

In specific embodiments, the first emulsifier may be selected from the group of quillaia, Arabic gum, octenyl succinic anhydride (OSA) modified waxy starch, a polysorbate, sucrose monostearate, a monoglyceride, soy lecithin, and Kolliphor RH40. In specific embodiments, the first emulsifier may be selected from the group of a polysorbate and Kolliphor RH40.

In specific embodiments, the first premixing stage may comprise: (i) combining and mixing the Cannabidiol, a (food-grade) oil, the first emulsifier, and optionally a further first premixing stage mixture component, and optionally (ii) subjecting the thus obtained first premixing stage mixture to a heating stage, wherein the first emulsifier at least comprises Kolliphor RH40, and wherein the heating stage comprises heating to a temperature of at least 30° C.

The term “the first emulsifier” may also refer to a plurality of different first emulsifiers.

According to BASF, https://pharma.basf.com/products/kolliphor-rh-40, Kolliphor RH 40 is a nonionic solubilizer and emulsifying agent obtained by reacting 40 moles of ethylene oxide with 1 mole of hydrogenated castor oil. The main constituent of Kolliphor RH 40 is glycerol polyethylene glycol oxystearate, which, together with fatty acid glycerol polyglycol esters, forms the hydrophobic part of the product. The hydrophilic part consists of polyethylene glycols and glycerol ethoxylate. Kolliphor RH 40 is a white to yellowish paste at 20° C. The monoglyceride may especially be a glycerin fatty acid ester.

In embodiments, the first premixing stage mixture may comprise the first emulsifier in a ratio of 1:4 relative to the food grade oil up to a ratio of 8:1 (in the first premixing stage mixture). In embodiments, the first premixing stage mixture may comprise the first emulsifier in a ratio of 1:2 relative to the food grade oil up to a ratio of 4:1 (in the first premixing stage mixture).

In embodiments, the food-grade oil may be selected from the group of Cannabis sativa oil, MCT oil, and sunflower oil.

In specific embodiments, the first premixing stage mixture may comprise Cannabidiol in an amount of 5-40 weight % (relative to the first premixing stage mixture). More especially, in specific embodiments the first premixing stage mixture may comprise Cannabidiol in an amount of 9-30 weight % (relative to the first premixing stage mixture).

Especially, the phrase “relative to the first premixing stage mixture” refers to the total weight of the first premixing stage mixture. Likewise this may apply to similar phrases.

In specific embodiments, the further first premixing stage mixture component may comprise ethanol, and the first premixing stage mixture may comprise ethanol in an amount of 5-85 weight %, such as 15-85 weight %, more especially in an amount of 25-75 weight % (relative to the first premixing stage mixture). In other embodiments, the further first premixing stage mixture component comprises ethanol, and the first premixing stage mixture comprises ethanol in an amount of 5-50 weight %, such as 10-50 weight % (relative to the first premixing stage mixture). In embodiments, the first premixing stage mixture comprises ethanol in an amount of 5-25 weight % (relative to the first premixing stage mixture).

In embodiments, the emulsifier may comprise a second emulsifier. Note that the presence of a second emulsifier does not necessarily include the presence of the first emulsifier.

Hence, in embodiments the components may comprise a second emulsifier.

Especially, in embodiments the first mixing stage may comprise a second premixing stage. The second premixing stage may comprise combining and mixing the second emulsifier and water to provide a second premixing stage mixture (“substantially aqueous liquid comprising premixing stage mixture”).

In specific embodiments, the second emulsifier may be selected from the group of quillaia, Arabic gum, octenyl succinic anhydride (OSA) modified waxy starch, a polysorbate, sucrose monostearate, a monoglyceride, soy lecithin, and Kolliphor RH40. For further embodiments in relation to the second emulsifier it is also referred to some of the embodiments described in relation to the first emulsifier.

In specific embodiments, the second emulsifier may be selected from the group of quillaia, Arabic gum, octenyl succinic anhydride modified waxy starch, sucrose monostearate, a monoglyceride, and soy lecithin. In further specific embodiments, the second emulsifier may be selected from the group of a sucrose monostearate and soy lecithin.

The term “the second emulsifier” may also refer to a plurality of different second emulsifiers.

In specific embodiments, the second premixing stage mixture may comprise the second emulsifier in an amount of 0.5-10 weight %, more especially 1-5 weight % (relative to the second premixing stage mixture).

As can be derived from the above, the first emulsifier may comprise one or more first emulsifiers and the second emulsifier may comprise one or more second emulsifiers. Especially, in embodiments the composition of the first emulsifier (comprising one or more first emulsifiers) is different from the composition of the second emulsifier (comprising one or more second emulsifiers).

In specific embodiments, the total weight percentage of polysorbate and Kolliphor RH40 in the first emulsifier is more than 50 wt %, and the total weight percentage of sucrose monostearate and soy lecithin in the second emulsifier is more than 50 wt %.

The phrase “total weight percentage of a and b”, and similar phrases refer to embodiments wherein one of these options a and b is available and to embodiments where both options a and b are available.

Therefore, in specific embodiments a first premixing stage mixture and a second premixing stage mixture may be provided. These two mixtures may be combined (and mixed).

Hence, in embodiments the first mixing stage may comprise: combining and mixing the first premixing stage mixture and the second premixing stage mixture, to provide the first mixture.

Therefore, in embodiments the first mixture may be processed into the first liquid via the first mixing stage and the first homogenization stage. However, in embodiments also the first mixture may be provided by combining the first premixing stage mixture and the second premixing stage mixture (which may have been subjected to a heat treatment). This first mixture may then be processed into the first liquid via the first mixing stage and the first homogenization stage.

In embodiments, the first premixing stage mixture and the second premixing stage mixture may be combined in a weight ratio between 1:99-25:75, more especially 5:95-20:80. Hence, the amount of the Cannabidiol comprising premixing stage mixture may be lower than the substantially aqueous liquid comprising premixing stage mixture.

In embodiments, the first mixture (or the first liquid) may comprise Cannabidiol in an amount of 0.1-10 weight %, such as especially 0.25-10 weight %, more especially in an amount of 0.5-6 weight % (relative to the first mixture). In other specific embodiments, the first mixture (or the first liquid) may comprise Cannabidiol in an amount of 0.25-4 weight %, more especially in an amount of 0.25-2 weight % (relative to the first mixture).

In embodiments, the first mixture (or the first liquid) comprises the oil in an amount of 0.15-40 weight %, such as in an amount of 0.3-20 weight % (relative to the first mixture (or the first liquid)). In embodiments, the first mixture (or the first liquid) comprises the oil in an amount of at least 1 weight % (relative to the first mixture (or the first liquid)).

In embodiments, the first mixture (or the first liquid) comprises the emulsifier in an amount of 0.1-50 weight %, such as in an amount of 0.25-30 weight % (relative to the first mixture (or the first liquid)). In embodiments, the first mixture (or the first liquid) comprises the emulsifier in an amount of at least 1 weight % (relative to the first mixture (or the first liquid)).

In embodiments, the first mixture (or the first liquid) comprises water in an amount of at least about 44 weight %, more especially at least 50 weight % (relative to the first mixture (or the first liquid)). In embodiments, the first mixture (or the first liquid) comprises water in an amount of at maximum about 99 weight % (relative to the first mixture (or the first liquid)).

Hence, in an aspect the invention provides a method for producing a liquid, wherein the method comprises a first stage for providing a first liquid comprising components, wherein the components comprise water, oil, an emulsifier, and a functional component, wherein the functional component comprises Cannabidiol, wherein the first stage comprises: (a) a first premixing stage, wherein the first premixing stage comprises: combining and mixing the Cannabidiol, oil, first emulsifier, and optionally a further first premixing stage mixture component, to provide a first premixing stage mixture; (b) a first mixing stage, wherein the first mixing stage comprises: combining and mixing water and the first premixing stage mixture to provide a first mixture, wherein in the first mixing stage a mixer device is applied rotating at a speed of at least 5000 RPM; and (c) a first homogenization stage to provide the first liquid, wherein the first homogenization stage comprises homogenizing in a homogenizer n times the first mixture, wherein in the first homogenization stage a homogenizer is applied at a pressure of at least 250 bar; wherein n is selected from the range of 1-10.

Hence, in an aspect, the invention provides a product (see also further below). Especially, in specific embodiments the product may comprise the first liquid, wherein the product comprises, relative to the total weight of the product: Cannabidiol in an amount of 0.5-6 weight %; the oil in an amount of 0.3-20 weight %; the emulsifier in an amount of 0.25-30 weight %; and wherein the product further comprises water, and optionally ethanol. Especially, the (first liquid) product may have a turbidity of not more than 500 Nephelometric Turbidity units.

Especially, in embodiments the first emulsifier and the second emulsifier are different (or comprise different compositions). Hence, when the first emulsifier comprises two or more emulsifiers and/or the second emulsifier comprises two or more emulsifiers, then in specific embodiments the first emulsifier and the second emulsifier comprise different compositions.

In embodiments, in the first mixing stage a mixer device may be applied rotating at a speed of at least 10.000 RPM, such as selected from the range of 10.000-25.000. However, other rotational speeds may also be possible.

Especially, in embodiments the mixer device for the first mixing stage may comprise an ultra-turrax device.

In embodiments, the mixer device for the first mixing stage may be a high shear mixing device. In embodiments, the mixing device may be a low pressure homogenizer, such as up to 15 bar, such as up to 10 bar, like at atmospheric pressure.

In embodiments, the mixer device for the first mixing stage may be of the rotor-stator type, such as especially an ultra-turrax device. In embodiments, the mixer device for the first mixing stage may be a rotor-stator homogenizer. Especially, the mixer device for the first mixing stage is a high shear batch mixer, and may comprise a rotating shaft in a stationary cylinder; hereby, drops may be reduced in size between the gap created between these two cylinders due to the shear forces that arise. Especially, the mixer device for the first mixing stage is based on the shear forces in the rotor-stator homogenizer. Such homogenizer may be a low pressure homogenizer, as the mixing device may in general be operated at ambient pressures, or at least below about 15 bar (see also above).

After the first mixing stage, the first homogenization stage may be executed. In embodiments, after the first mixing stage, and before the first homogenization stage, there may be a resting period. In general, the resting period may be selected from the range of 5 minutes to 300 minutes, such as up to about 120 minutes. A longer resting period is possible, but may be less desirable. The resting period may promote emulsion formation and/or stability.

In specific embodiments, in the first homogenization stage a homogenizer may be applied at a pressure of at least 500 bar.

Good results were obtained when n may be selected from the range of 2-10, such as 2-6, such as in specific embodiments at least 3, like e.g. 3-5. In embodiments, n may be selected from the range of 3-10. In specific embodiments, n may be selected from the range of 3-6.

There are many different types and models of homogenizers depending on the material to be processed. Suitable for homogenization of milk is usually a high-pressure homogenizer machine. This electric milk homogenizing equipment is actually a three-plunger reciprocating pump, which is mainly composed of the main drive shaft, a transmission belt, a body, a seal, a plunger, a suction valve, a homogenizing valve, a valve stem, a pressure gauge, a discharge valve, and the like.

Herein, the homogenizer used for the first homogenization stage may especially be a high pressure homogenizer, such as using a pressure of at least 250 bar (during the homogenization stage).

Especially, the homogenizer used for the first homogenization stage may be a high pressure homogenizer. Such high pressure homogenizer may comprise a high shear continuous pump. In embodiments, the homogenizer used for the first homogenization stage may be a high pressure pump where at a pressure of typically hundreds to thousands of bars, such as especially at least about 250 bar in the present invention, a liquid is forced through a narrow gap and droplets are broken up by the resulting shear forces.

In the present invention, however, the pressure (used in the homogenization stage) may be substantially higher than for the homogenization for milk.

In specific embodiments, during each homogenization (of the n homogenizations) the elevated pressure may be applied during a time period selected from the range of 0.05-60 seconds, more especially 0.1-20 seconds.

With the homogenizer in the first homogenization stage particles may be created in the submicron range. In embodiments, cannabidiol comprising particles may be provided in the first homogenization stage having particle sizes below 500 nm, such as even below 200 nm. In embodiments, at least 50 vol. % of the cannabidiol comprising particles may have particle sizes selected from the range of up to about 200 nm, such as even up to about 100 nm. The small droplets may improve intake in the human body of the functional component. The small droplets may in embodiments essentially be oil droplets comprising the functional component. Hence, the functional component may especially be comprised by an oil phase. In embodiments, particle sizes may be determined using laser scattering.

In specific embodiments, the components may further comprise one or more of citric acid, ascorbic acid, and sodium benzoate. These may be added for e.g. conservation purposes. Such component(s) may be added before or after homogenization. In embodiments, such component(s) may be added before homogenization.

The thus obtained first liquid may thus be turbid, but may comprise a relatively high concentration of the functional component.

In embodiments, the invention also provides the first mixture per se.

The first liquid may thus essentially be the first mixture after the first mixing stage and the first homogenization stage.

Hence, a first mixture may be obtained as such due to combining and mixing the components during a first mixing stage, whereafter the first mixture is homogenized in the first homogenization stage, to provide a homogenized first mixture.

A first mixture may also be obtained by executing the first premixing stage and the second premixing stage, and combining and mixing the first premixing stage mixture and the second stage mixture to provide the first mixture in the first mixing stage, i.e. especially wherein the mixer device is applied rotating at a speed of at least 5000 RPM, whereafter the first mixture is homogenized in the first homogenization stage, to provide a homogenized first mixture.

Especially, the term “first liquid” may refer to the first mixture after the first homogenization stage.

In embodiments, the thus obtained first liquid may be diluted to provide a second liquid, or may be concentrated for later use in a product. Embodiments thereof are described below.

In embodiments, the method may (further) comprise a second stage for providing a second liquid comprising the first liquid. Especially, the second stage may comprise a second mixing stage. The second mixing stage may comprise combining and mixing the first liquid and an aqueous liquid, to provide the second mixture. This mixing and combining may be done with methods known in the art, and may not necessarily include high shear mixing methods as described above in relation to the first mixing stage and/or first homogenization stage.

The second liquid may thus essentially be the second mixture, which may essentially be a dilution of the first liquid (or first mixture after the first mixing stage and the first homogenization stage).

Especially, the aqueous liquid may be water. In other embodiments, the aqueous liquid may comprise water and ethanol.

In this way, oil droplets comprising the functional component may be comprised in aqueous (continuous) phase.

In specific embodiments, (i) the first liquid may be selected such and (ii) the second stage may be executed such that the second liquid has a turbidity of not more than 50 Nephelometric Turbidity units. More especially, in embodiments (i) the first liquid may be selected such and (ii) the second stage may be executed such that the second liquid has a turbidity of not more than 25 Nephelometric Turbidity units.

In embodiments, the second mixing stage may comprise diluting the first liquid with the aqueous liquid in the order of 50-4000 times, more especially 100-2000 times, to provide the second mixture.

In specific embodiments, the second mixture may comprise Cannabidiol in an amount of 0.0005-0.06 weight %, such as especially 0.0005-0.04 weight %, more especially 0.001-0.02 weight % (relative to the second mixture).

In specific embodiments, the second mixture may comprise the oil in an amount of 0.0015-0.4 weight %, more especially 0.003-0.2 weight % (relative to the second mixture).

In specific embodiments, the second mixture may comprise the emulsifier in an amount of 0.0015-0.4 weight %, such as in embodiments 0.0015-0.2 weight %, like especially 0.0015-0.2 weight %, more especially 0.0025-0.1 weight % (relative to the second mixture). Note that here the term “emulsifier” may thus especially refer to a combination of the first emulsifier and the second emulsifier.

In embodiments, the invention also provides the second mixture (or second liquid) per se.

Hence, in specific embodiments the product may comprise the second liquid, wherein the product comprises, relative to the total weight of the product: cannabidiol in an amount of 0.001-0.06 weight %; the oil in an amount of 0.003-0.2 weight %; the emulsifier in an amount of 0.0025-0.3 weight %; and wherein the product further comprises water, and optionally ethanol. Further, the (second liquid) product may have a turbidity of not more than 25 Nephelometric Turbidity units.

In embodiments, the invention also provides the first liquid per se (see also above).

In embodiments, the method for producing the first liquid may be followed by a spray drying stage. Hence, in embodiments the method may further comprise a spray drying stage. Especially, the spray drying stage may comprise combining and mixing the first mixture with an additive comprising one or more of a water soluble protein and a water soluble carbohydrate, and spray drying the thus obtained mixture to provide a spray dried product.

In specific embodiments, the additive may comprise one or more of maltodextrin, pea protein, and soy protein. However, other additives are herein not excluded. Further, combinations of additives may also be possible.

Hence, in embodiments the spray drying stage may comprise combining and mixing the first mixture with an additive comprising maltodextrin, and spray drying the thus obtained mixture to provide a spray dried product. In other embodiments, the spray drying stage may comprise combining and mixing the first mixture with an additive comprising (i) a water soluble protein and (ii) a water soluble carbohydrate, and spray drying the thus obtained mixture to provide a spray dried product.

In embodiments, the method may comprise spray drying at a temperature selected from the range of 80-175° C.

In embodiments, the spray dried product may have less than 15 weight % water (relative to the spray dried product). Especially, in embodiments the spray dried product may have 5-8 wt % water.

In embodiments, the invention also provides the spray dried product per se.

The spray dried product may in embodiments comprise Cannabidiol in an amount of 0.5-8 weight %, more especially 1-7 weight % (relative to the total weight of the spray dried product).

The spray dried product may in embodiments comprise the oil in an amount of 0.25-40 weight %, more especially 0.5-25 weight % (relative to the total weight of the spray dried product).

The spray dried product may in embodiments comprise the emulsifier in an amount of 0-25-50 weight %, more especially 0.5-40 weight % (relative to the total weight of the spray dried product).

The spray dried product may in embodiments comprise the additive, comprising one or more of a water soluble protein and a water soluble carbohydrate, in an amount of 15-95 weight %, more especially 20-95 weight % (relative to the total weight of the spray dried product).

Hence, in embodiments the product comprises the spray dried product, wherein the product comprises, relative to the total weight of the product:
Cannabidiol in an amount of 1-7 weight %;
the oil in an amount of 0.5-25 weight %;
the emulsifier in an amount of 0.5-40 weight %;
the additive, comprising one or more of a water soluble protein and a water soluble carbohydrate, in an amount of 20-95 weight %, and
less than 15 weight % water.

After spray drying, the spray dried product may be stored and/or transported, etc. For use of the spray dried product, the product may be used as such, or may be diluted with water to provide effectively the above defined second liquid.

Hence, in specific embodiments the method may comprise a third stage for providing a (the) second liquid. Especially, the third stage may comprise: a third mixing stage. In embodiments, the third mixing stage may comprise combining and mixing the spray dried product and an aqueous liquid, to provide the second mixture.

Especially, the aqueous liquid may be water. In other embodiments, the aqueous liquid may comprise water and ethanol.

In an aspect, the invention also provides the use of the second liquid as defined herein, or (the use of) the spray dried product as defined herein, for the preparation of a medicament or a functional food. In yet another aspect, the invention also provides the use of the first liquid as defined herein, for the preparation of a medicament or a functional food.

In an aspect, the invention provides a product comprising one or more of the first liquid obtainable with the method(s) as described herein, or the second liquid obtainable according to the method(s) as described herein, or the spray dried product obtainable with the method(s) as described herein.

In an aspect, the invention also provides a product selected from the group of a medicament and a functional food. Such product may comprise one or more of the first liquid obtainable with the method(s) as described herein, or the second liquid obtainable according to the method(s) as described herein, or the spray dried product obtainable according to the method(s) as described herein, for use in a medical treatment. Instead of the term “medicament” also the term “pharmaceutical composition” may be used.

The product may be used for the treatment of anxiety, movement disorders, and pain. The product may be used for the promotion of cognition. The product may be used for the treatment of epilepsy disorders. The product may be used for the treatment of spasm (i.e. especially a sudden involuntary contraction of a muscle).

In yet a further aspect, the invention also provides an arrangement comprising (i) a mixer device and (ii) a homogenizer, wherein the latter is configured downstream to the former. Especially, the mixer device is an ultra-turrax.

In yet a further aspect the invention also provides an arrangement comprising (i) a mixer device and (ii) a homogenizer, wherein the homogenizer is configured downstream to the former, as well as (iii) a second mixing device, wherein the second mixing device is configured downstream of the homogenizer. The second mixing device may be configured to dilute the first liquid with the aqueous liquid.

In yet a further aspect the invention also provides an arrangement comprising (i) a mixer device and (ii) a homogenizer, wherein the homogenizer is configured downstream to the former, as well as (iii) a spray dryer device, wherein spray dryer device is configured downstream of the homogenizer.

Amongst others, the invention provides in an aspect (and in embodiments) a production process by which a CBD in water solution can be made with a turbidity of 50 NTU or lower by successively adding the following production steps:
an emulsifying system A is mixed with water;
an emulsifying system B is mixed with a food-grade oil containing CBD dissolved (CBD oil) in a percentage of between 5% and 40% CBD in the relevant food-grade oil. This oil mixture is then heated between 2° and 60° C. and then cooled again;
the water with emulsifying system A and the CBD oil with emulsifying system B are mixed in a ratio between 95%-5% or 80%-20% by mass, and then this mixture is homogenized with a high shear mixer (ultra turax) at a speed of at least 10,000 RPM for at least 1 minute;
the homogenized water and oil mixture from the above step is then treated between 2 and 6 times with a high-pressure homogenizer at a pressure between 500 and 1200 bar.

In embodiments, the high pressure homogenized mixture is diluted with water by a factor between 100 times and 2000 times making the turbidity less than 50 NTU and preferably less than 25 NTU.

In embodiments, the emulsifying system A may consist of a mixture comprising sucrose monostearate and/or lecithin in a ratio between 100:0 and 0:100 in a concentration between 2.5% and 15% by mass. (Base in the water).

In embodiments, the emulsifying system B may consist of a mixture comprising Kolliphor RH40 and/or polysorbate in the CBD oil.

In embodiments, the food-grade oil from the second production step may be hemp oil, MCT oil, and/or sunflower oil; optionally mixed with a fraction of ethanol. In embodiments, the CBD concentration in this food-grade oil may be lower than 30% but also higher than 9%.

In embodiments, the homogenization pressure may be between 750 and 1000 bar and the number of high pressure treatments may be between 3× and 5×.

In embodiments, a preservative such as citric acid, sodium benzoate, and/or ascorbic acid may be (finally) added to the high-pressure homogenized water-oil mixture.

In embodiments, a vegetable protein concentrate or isolate (for example from pea, soy, or wheat) or a carbohydrate (for example maltodextrin) may be added to the high-pressure homogenized water, and the (thus obtained) product may then spray dried to a powder.

### BRIEF DESCRIPTION OF THE DRAWINGS

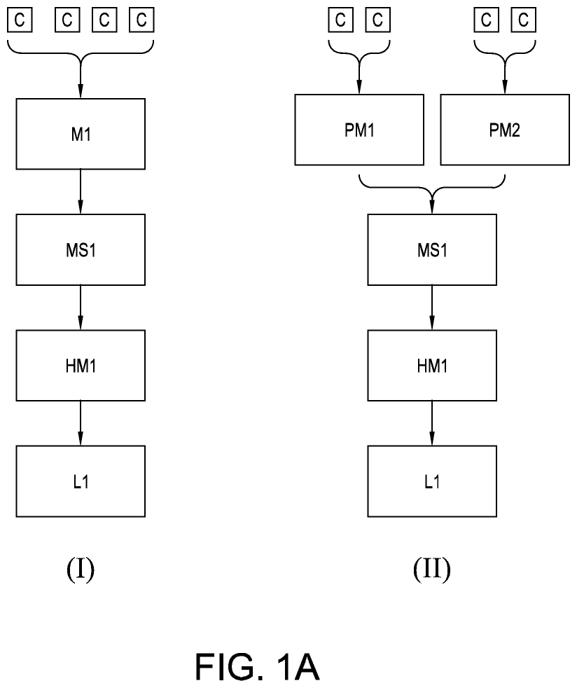

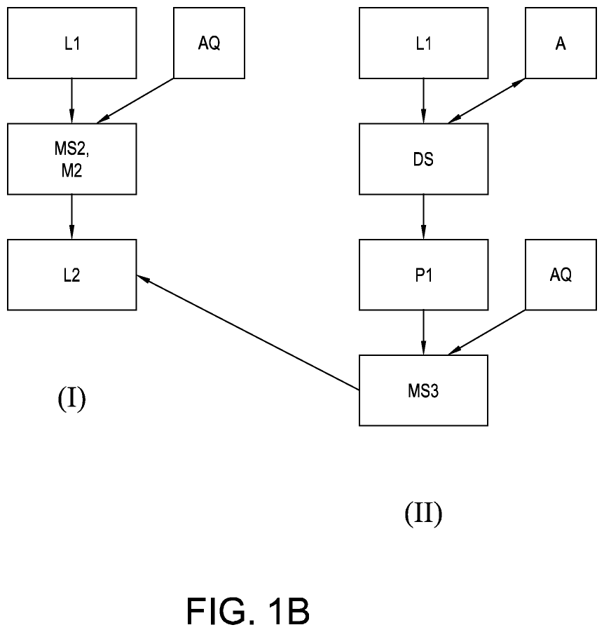

Embodiments of the invention will now be described, by way of example only, with reference to the accompanying schematic drawings in which corresponding reference symbols indicate corresponding parts, and in which: FIGS. 1a-1b schematically depict some aspects of the invention. The schematic drawings are not necessarily to scale.

### DETAILED DESCRIPTION OF THE EMBODIMENTS

### Homogenizer Protocol

A stable, optically clear, beverage is desirably obtained. Homogenization is carried out with a pressure of between 300 bar and 1500 bar, preferably about 800 bar. The homogenization pressure may be applied for between 1 and 5 passes. The ratio of emulsifier to carrier oil in the emulsion may be containing an excess amount of emulsifier in oil, which could be used to make an optically clear beverage. An optimal concentration of between 0 and 3 times more emulsifier in contrast to the oil phase will be tested. Cloudy beverages have a high turbidity, e.g., between 200 and 300 NTU, or even as high as 2,000 NTU. The aim is to develop a beverage with a turbidity<15 NTU. After production of the treatments, the turbidity will therefore be measured by a turbidity meter.

### Emulsifiers

Based on literature and information from previous experiments, a list composed of “to be tested” emulsifiers are shown in the below table 1:

TABLE 1
List of emulsifiers that will be tested in the lab.
Emulsifiers

Quillaia (Q-naturale)
Gum Arabic
OSA-starch
Polysorbate 20/Tween 20 and polysorbate 80/Tween 80
Sucrose monostearate (se, HLB 15)
Monoglyceride
Soy lecithin
Kolliphor RH 40

### Protocol

1. Before starting the experiment, the most optimal solubilizing phase of the emulsifier (organic or in aqueous phase) may be investigated. This could influence the droplet size and is different for every emulsifier (the movement of the emulsifier from the oil phase into the aqueous phase may be important in the formation of nano emulsions by this method).
2. Prepare a pre-emulsion comprising the emulsifier in water or oil phase in order to fully dissolve the emulsifier. When mixing the emulsifier into the solubilizing phase, an Ultra turrax is used at 10000 rpm (stand 37) for 3 minutes. If needed, the emulsifier and solubilized phase could be heated prior to mixing.
3. Mix the pre-emulsion by slowly adding the oil phase to the aqueous phase while mixing with an Ultra turrax at 12000 rpm (stand 40) for 5 min).
4. Let the pre-emulsion mixture rest for 1 hour.
5. Homogenize the emulsion with a homogenizer preferably between 300-1200 bar, and between 3 and 5 passes.
6. Collect the sample and measure the temperature directly.
7. Allow it to cool completely before measuring the turbidity.
8. The concentrated emulsion may especially be made into a ready-to-drink clear beverage by diluting it with water. Therefore, dilute a small amount of the obtained concentrated emulsion from step 6 with water to observe the clarity and turbidity of the final product. For 5% of oil, the dilution factor will be 165×.
9. Measure the turbidity before and after dilution.
10. Take a picture of the solution before and after dilution.
11. Taste the diluted emulsion for bitterness or other off flavors.
12. Store and look at the shelf life.

### Experimental Design

Emulsifier type and concentration, emulsifier to oil ratio, addition of essential oil to oil phase and applied pressure are the experimental parameters that will be tested. They are divided over experimental Trial X1 (Table 2) and Trial X2 (Table 3).

TABLE 2
Experimental design of Trial X1.

| | | | | Bar |
|---|---|---|---|---|
| Experimental | | | | (homogenizor |
| variable | Treatment description | Treatment | Emulsifier type | pressure) |
| Applied pressure | Emulsifier:oil (3 × 2:1) | 1A | Tween 20 + OSA starch | 300 |
| (Bar) | Emulsifier:oil (3 × 2:1) | 1B | Tween 20 + OSA starch | 800 |
| | Emulsifier:oil (3 × 2:1) | 1C | Tween 20 + OSA starch | 1000 |
| Concentration | Emulsifier:oil (1 × 2:1) + | 2A | Tween 80 + OSA starch | 800 |
| ratio | Becanex 20% CBD oil | | | |
| emulsifiers | Emulsifier:oil (2 × 2:1) + | 2B | Tween 80 + OSA starch | 800 |
| | Becanex 20% CBD oil | | | |
| | Emulsifier:oil (3 × 2:1) + | 20 | Tween 80 + OSA starch | |
| | Becanex 20% CBD oil | | | |
| | Emulsifier oil (3.5:1) | 2D | Tween 80 + OSA starch | 800 |
| | (tween80 = 0.5, OSA starch = | | | |
| | 3) + Becanex 20% CBD oil | | | |
| Concentration | Emulsifier:oil (1:1) + Becanex | 3A | OSA starch | 800 |
| ratio | 20% CBD oil | | | |
| emulsifiers | Emulsifier:oil (2:1) + normal | 3B | OSA starch | 800 |
| | MCT oil with no CBD | | | |
| | Emulsifier oil (3:1) + Becanex | 3C | OSA starch | |
| | 20% CBD oil | | | |
| | Emulsifier:oil (5:1) + Becanex | 3D | OSA starch | 800 |
| | 20% CBD oil | | | |
| Concentration | Emulsifier:oil (Ratio from | 4A | Q-naturale | 800 |
| emulsifier | Ingredion: 1:4) | | | |
| | Emulsifier oil (1:1) | 4B | Q-naturale | 800 |
| | Emulsifier:oil (2:1) | 4C | Q-naturale | 800 |
| | Emulsifier:oil (3:1) | 4D | Q-naturale | 800 |
| Concentration | Emulsifier:oil (from literature: | 5A | Sucrose monostearate | 800 |
| emulsifier | 0.1:1) | | | |
| | Emulsifier:oil (1:1) | 5B | Sucrose monostearate | 800 |
| | Emulsifier:oil (0.5:1) | 5C | Sucrose monostearate | 800 |
| Concentration | Emulsifier:oil (2:1) | 6A | Monoglyceride | 800 |
| emulsifier | Emulsifier:oil (3:1) | 6B | Monoglyceride | 800 |
| | Emulsifier:oil (1:1) | 6C | Monoglyceride | 800 |
| Concentration | Emulsifier:oil (3:1) + addition | 7A | 85% Tween 20 + 15% | 800 |
| ratio | of a cosurfactant | | lecithine | |
| emulsifiers | Emulsifier:oil (3:1) + addition | 7B | 65% Tween 20 + 35% | 800 |
| | of a cosurfactant | | lecithine | |
| | Emulsifier:oil (3:1) + addition | 7C | 100% Tween20 + lecithine | 800 |
| | of a cosurfactant | | (1% of total) | |
| Concentration | Emulsifier:oil (1:1) based on | 8A | Arabic gum | 800 |
| emulsifier | literature | | | |
| | Emulsifier:oil (3:1) | 8B | Arabic gum | 800 |
| Ratio essential | Essential oil:MCT (4:1) | 9A | Q-naturale (ratio from 4A) | 800 |
| oil:MCT | Essential oil:MCT (1:4) | 9B | Q-naturale (ratio from 4A) | 800 |
| | Essential oil:MCT (1:1) | 9C | Q-naturale (ratio from 4A) | 800 |
| Ratio essential | Essential oil:MCT (4:1) + | 10A | 65% Tween 20 + 35% | 800 |
| oil:MCT | Emulsifier:oil (1:1) | | lecithine | |
| | Essential oil:MCT (1:4) + | 10B | 65% Tween 20 + 35% | 800 |
| | Emulsifier:oil (1:1) | | lecithine | |
| | Essential oil:MCT (1:1) + | 10C | 65% Tween 20 + 35% | 800 |
| | Emulsifier:oil (1:1) | | lecithine | |

TABLE 3
Experimental design set-up of Trial X2.

| Experimental | Treatment | | | Pressure |
|---|---|---|---|---|
| variable | description | Treatment | Emulsifier type | (Bar) |
| Emulsifer:oil ratio | Emulsifier:oil (1:1) | 1A | 65% Tween 20 + 35% lecithine | 800 |
| | Emulsifier:oil (2:1) | 1B | 65% Tween 20 + 35% lecithine | 800 |
| | Emulsifier:oil (3:1) | 1C | 65% Tween 20 + 35% lecithine OR | 800 |
| | | | span80 | |
| Applied pressure | Emulsifier:oil (1:1) | 2A | 65% Tween 20 + 35% lecithine OR | 300 |
| (Bar) | | | span80 | |
| | Emulsifier:oil (1:1) | 2B | 65% Tween 20 + 35% lecithine OR | 1200 |
| | | | span80 | |
| | Emulsifier:oil (1:1) | 2C | 65% Tween 20 + 35% lecithine OR | 1500 |
| | | | span80 | |
| Emulsifier type + | Emulsifier:oil (0.5:1) | 3A = 5C- | Sucrose mono stearate | 800 |
| concentration | | X1 | | |
| | Emulsifier:oil (0.75:1) | 3B | Sucrose mono stearate | 800 |
| | Emulsifier:oil (0.6:1) | 3C | 90% sucrose monostearate + 10% | 800 |
| | | | monoglyceride | |
| Emulsifiers mixed | Emulsifier:oil (1:1) | 4A | 50% sucrose monostearate + 50% | 800 |
| | | | tween 80 | |
| | Emulsifier:oil (1.15:1) | 4B | 50% sucrose monostearate + 65% | 800 |
| | | | tween 80 | |
| | Emulsifier:oil (1.1:1) | 4C | 50% sucrose monostearate + 50% | 800 |
| | | | tween 80 + (1%*total amount | |
| | | | lecithine) | |
| | Emulsifier:oil (1.7:1) | 4D | 29.4% sucrose monostearate + | 800 |
| | | | 29.4% tween 80 + 41.18% g | |
| | | | lecithine | |
| | Emulsifier:oil (1.7:1) | 4E | 29.4% sucrose monostearate + | 800 |
| | | | 17.6% tween 80 + 52.9% lecithine | |
| Emulsifier Type | Emulsifier:oil (1:1) | 5A | 21.7% Tween 80 + 35% lecithine | 800 |
| | Emulsifier:oil (2:1) | 5B | 21.7% Tween 80 + 35% lecithine | 800 |
| | Emulsifier:oil (3:1) | 5C | 21.7% Tween 80 + 35% lecithine | 800 |
| with CBD in | Emulsifier:oil (0.5:1) | 6A = 5C- | sucrose monostearate | 800 |
| oil. Feb.11, 2021 | with 20% CBD from | X1 | | |
| | Becanex | | | |
| | Emulsifier:oil (1.1:1) | 6B = 4C- | 50% sucrose monostearate + 50% | 800 |
| | with 20% CBD from | X2 | tween 80 + (1%*total lecithine) | |
| | Becanex | | | |
| | Emulsifier:oil (1.1:1) | 6D = 4C- | 50% sucrose monostearate + 50% | 800 |
| | with 5% CBD from | X2 | tween 80 + (1%*total lecithine) | |
| | Becanex | | | |
| | Emulsifier:oil (1.1:1) | 6C = 4C- | 50% sucrose monostearate + 50% | 800 |
| | with 40% CBD | X2 - 500 mL | tween 80 + (1%*total lecithine) | |
| Emulsifier | From Literature = | 7A | Kolliphor RH 40 | 800 |
| concentration | emulsifier:oil (9:1) | | | |
| | Emulsifier:(oil + | 7B | Kolliphor RH40 + ethanol | 800 |
| | ethanol (2:1)) (2:1) | | | |
| | Emulsifier:oil (1:1) | 7C | Kolliphor RH 40 | 800 |
| | Emulsifier:oil (2:1) | 7D | Kolliphor RH 40 | 800 |
| | Emulsifier:oil (3:1) | 7E | Kolliphor RH 40 | 800 |

Trial X1: table first part

| Experimental | | | |
|---|---|---|---|
| variable | Treatment description | Treatment | Emulsifier type |
| Applied | Emulsifier:oil (3 × 2:1) | 1A | Tween 20 + OSA starch |
| pressure (Bar) | Emulsifier:oil (3 × 2:1) | 1B | Tween 20 + OSA starch |
| | Emulsifier:oil (3 × 2:1) | 1C | Tween 20 + OSA starch |
| Concentration | Emulsifier:oil (1 × 2:1) + Becanex | 2A | Tween 80 + OSA starch |
| ratio | 20% CBD oil | | |
| emulsifiers | Emulsifier:oil (2 × 2:1) + Becanex | 2B | Tween 80 + OSA starch |
| | 20% CBD oil | | |
| | Emulsifier:oil (3 × 2:1) + Becanex | 2C | Tween 80 + OSA starch |
| | 20% CBD oil | | |
| | Emulsifier oil (3.5:1) | 2D | Tween 80 + OSA starch |
| | (tween80 = 0.5, OSA starch = 3) + | | |
| | Becanex 20% CBD oil | | |
| Concentration | Emulsifier:oil (1:1) + Becanex | 3A | OSA starch |
| ratio | 20% CBD oil | | |
| emulsifiers | Emulsifier:oil (2:1) + normal MCT | 3B | OSA starch |
| | oil with no CBD | | |
| | Emulsifier oil (3:1) + Becanex | 3C | OSA starch |
| | 20% CBD oil | | |
| | Emulsifier:oil (5:1) + Becanex | 3D | OSA starch |
| | 20% CBD oil | | |
| Concentration | Emulsifier:oil (Ratio from | 4A | Q-naturale |
| emulsifier | Ingredion: 1:4) | | |
| | Emulsifier oil (1:1) | 4B | Q-naturale |
| | Emulsifier:oil (2:1) | 4C | Q-naturale |
| | Emulsifier:oil (3:1) | 4D | Q-naturale |
| Concentration | Emulsifier:oil (from literature: | 5A | Sucrose monostearate |
| emulsifier | 0.1:1) | | |
| | Emulsifier:oil (1:1) | 5B | Sucrose monostearate |
| | Emulsifier:oil (0.5:1) | 5C | Sucrose monostearate |
| Concentration | Emulsifier:oil (2:1) | 6A | Monoglyceride |
| emulsifier | Emulsifier:oil (3:1) | 6B | Monoglyceride |
| | Emulsifier:oil (1:1) | 6C | Monoglyceride |
| Concentration | Emulsifier:oil (3:1) + addition of a | 7A | 85% Tween 20 + 15% lecithin |
| ratio | cosurfactant | | |
| emulsifiers | Emulsifier:oil (3:1) + addition of a | 7B | 65% Tween 20 + 35% lecithin |
| | cosurfactant | | |
| | Emulsifier:oil (3:1) + addition of a | 7C | Tween20 + (1% of lecithin = |
| | cosurfactant | | 1%*300 g) |
| Concentration | Emulsifier:oil (1:1) based on | 8A | Arabic gum |
| emulsifier | literature | | |
| | Emulsifier:oil (3:1) | 8B | Arabic gum |
| Ratio essential | Essential oil:MCT (4:1) | 9A | Q-naturale (ratio from 4A) |
| oil:MCT | Essential oil:MCT (1:4) | 9B | Q-naturale (ratio from 4A) |
| | Essential oil:MCT (1:1) | 9C | Q-naturale (ratio from 4A) |
| Ratio essential | Essential oil:MCT (4:1) + | 10A | 65% Tween 20 + 35% lecithin |
| oil:MCT | Emulsifier:oil (1:1) | | |
| | Essential oil:MCT (1:4) + | 10B | 65% Tween 20 + 35% lecithin |
| | Emulsifier:oil (1:1) | | |
| | Essential oil:MCT (1:1) + | 10C | 65% Tween 20 + 35% lecithin |
| | Emulsifier:oil (1:1) | | |

Trial X1: table second part

| | | | | | | | Passes | |
|---|---|---|---|---|---|---|---|---|
| | | | | | | | (start | |
| | Oil | Concentration | | | | | with 3 | Bar |
| | (%) | Emulsifier | Water | Oil | Emulsifier | Water | up to | (homogenisator |
| Treatment | (MCT) | (%) | (%) | (g) | (g) | (g) | 5x) | pressure) |
| 1A | 5% | 30.00% | 65.00% | 10 | 60 | 130 | 5 | 300 |
| 1B | 5% | 30.00% | 65.00% | 10 | 60 | 130 | 5 | 800 |
| 1C | 5% | 30.00% | 65.00% | 10 | 60 | 130 | 5 | 1000 |
| 2A | 5% | 10.00% | 85.00% | 10 | 20 | 170 | 5 | 800 |
| 2B | 5% | 20.00% | 75.00% | 10 | 40 | 150 | 5 | 800 |
| 2C | 5% | 30.00% | 65.00% | 10 | 60 | 130 | 5 | 800 |
| 2D | 5% | 17.50% | 77.50% | 10 | 35 | 155 | 5 | 800 |
| 3A | 5% | 5.00% | 90.00% | 10 | 10 | 180 | 5 | 800 |
| 3B | 5% | 10.00% | 85.00% | 10 | 20 | 170 | 5 | 800 |
| 3C | 5% | 15.00% | 80.00% | 10 | 30 | 160 | 5 | 800 |
| 3D | 5% | 25.00% | 70.00% | 10 | 50 | 140 | 5 | 800 |
| 4A | 5% | 1.25% | 93.75% | 10 | 2.5 | 187.5 | 5 | 800 |
| 4B | 5% | 5.00% | 90.00% | 10 | 10 | 180 | 5 | 800 |
| 4C | 5% | 10.00% | 85.00% | 10 | 20 | 170 | 5 | 800 |
| 4D | 5% | 15.00% | 80.00% | 10 | 30 | 160 | 5 | 800 |
| 5A | 5% | 0.50% | 94.50% | 10 | 1 | 189 | 5 | 800 |
| 5B | 5% | 5.00% | 90.00% | 10 | 10 | 180 | 5 | 800 |
| 5C | 5% | 2.50% | 92.50% | 10 | 5 | 185 | 5 | 800 |
| 6A | 5% | 10.00% | 85.00% | 10 | 20 | 170 | 5 | 800 |
| 6B | 5% | 15.00% | 80.00% | 10 | 30 | 160 | 5 | 800 |
| 6C | 5% | 5.00% | 90.00% | 10 | 10 | 180 | 5 | 800 |
| 7A | 5% | 15.00% | 80.00% | 10 | 30 | 160 | 5 | 800 |
| 7B | 5% | 15.00% | 80.00% | 10 | 30 | 160 | 5 | 800 |
| 7C | 5% | 15.00% | 80.00% | 10 | 30 | 160 | 5 | 800 |
| 8A | 5% | 5.00% | 90.00% | 10 | 10 | 180 | 5 | 800 |
| 8B | 5% | 15.00% | 80.00% | 10 | 30 | 160 | 5 | 800 |
| 9A | 5% | 1.25% | 93.75% | 10 | 2.5 | 187.5 | 5 | 800 |
| 9B | 5% | 1.25% | 93.75% | 10 | 2.5 | 187.5 | 5 | 800 |
| 9C | 5% | 1.25% | 93.75% | 10 | 2.5 | 187.5 | 5 | 800 |
| 10A | 5% | 5.00% | 90.00% | 10 | 10 | 180 | 5 | 800 |
| 10B | 5% | 5.00% | 90.00% | 10 | 10 | 180 | 5 | 800 |
| 10C | 5% | 5.00% | 90.00% | 10 | 10 | 180 | 5 | 800 |

Trial X2 table first part:

| Experimental | | | |
|---|---|---|---|
| variable | Treatment description | Treatment | Emulsifier type |
| Emulsifier:oil | Emulsifier:oil (1:1) | 1A | 65% Tween 20 + 35% lecithin OR |
| ratio | | | span80 |
| | Emulsifier:oil (2:1) | 1B | 65% Tween 20 + 35% lecithin OR |
| | | | span80 |
| | Emulsifier:oil (3:1) | 1C | 65% Tween 20 + 35% lecithin OR |
| | | | span80 |
| Applied pressure | Emulsifier:oil (1:1) | 2A | 65% Tween 20 + 35% lecithin OR |
| (Bar) | | | span80 |
| | Emulsifier:oil (1:1) | 2B | 65% Tween 20 + 35% lecithin OR |
| | | | span80 |
| | Emulsifier:oil (1:1) | 2C | 65% Tween 20 + 35% lecithin OR |
| | | | span80 |
| Emulsifier type + | Emulsifier:oil (0.5:1) | 3A = 5C-X1 | sucrose mono stearate |
| concentration | Emulsifier:oil (0.75:1) | 3B | sucrose mono stearate |
| | Emulsifier:oil (0.6:1) | 3C | 90% sucrose monostearate + 10% |
| | | | monoglyceride |
| Emulsifiers | Emulsifier:oil (1:1) | 4A | 50% sucrose monostearate + 50% |
| mixed | | | tween 80 |
| | Emulsifier:oil (1.15:1) | 4B | 50% sucrose monostearate + 65% |
| | | | tween 80 |
| | Emulsifier:oil (1.1:1) | 4C | 50% sucrose monostearate + 50% |
| | | | tween 80 + (1%*total amount |
| | | | lecithin) |
| | Emulsifier:oil (1.7:1) | 4D | 29.4% sucrose monostearate + |
| | | | 29.4% tween 80 + 41.18% g lecithin |
| | Emulsifier:oil (1.7:1) | 4E | 29.4% sucrose monostearate + |
| | | | 17.6% tween 80 + 52.9% lecithin |
| Emulsifier Type | Emulsifier:oil (1:1) | 5A | 21.7% Tween 80 + 35% lecithin |
| | Emulsifier:oil (2:1) | 5B | 21.7% Tween 80 + 35% lecithin |
| | Emulsifier:oil (3:1) | 5C | 21.7% Tween 80 + 35% lecithin |
| with CBD in oil. | Emulsifier:oil (0.5:1) with | 6A = 5C-X1 | sucrose monostearate |
| Feb. 11, 2021 | 20% CBD from Becanex | | |
| | Emulsifier:oil (1.1:1) with | 6B = 4C-X2 | 50% sucrose monostearate + 50% |
| | 20% CBD from Becanex | | tween 80 + (1%*total lecithin) |
| | Emulsifier:oil (1.1:1) with | 6D = 4C-X2 | 50% sucrose monostearate + 50% |
| | 5% CBD from Becanex | | tween 80 + (1%*total lecithin) |
| | Emulsifier:oil (1.1:1) with | 6C = 4C- | 50% sucrose monostearate + 50% |
| | 40% CBD | X2 - 500 mL | tween 80 + (1%*total lecithin) |
| Emulsifier | From Literature = | 7A | Kolliphor RH 40 |
| concentration | emulsifier:oil (9:1) | | |
| | emulsifier:(oil + ethanol | 7B | Kolliphor RH40 with ethanol |
| | (2:1)) (2:1) | | |
| | Emulsifier:oil (1:1) | 7C | Kolliphor RH 40 |
| | Emulsifier:oil (2:1) | 7D | Kolliphor RH 40 |
| | Emulsifier:oil (3:1) | 7E | Kolliphor RH 40 |
| With CBD in oil | Emulsifier:oil (2:1) | 8A | Kolliphor RH40 + 5% CBD |
| | Emulsifier:oil (2:1) | 8B | Kolliphor RH40 + 20% CBD |

Trial X2 table second part:

| | | | | | | | Passes | |
|---|---|---|---|---|---|---|---|---|
| | | | | | | | (start | |
| | Oil | Concentration | | | | | with 3 | Bar |
| | (%) | Emulsifier | Water | Oil | Emulsifier | Water | up to | (homogenisator |
| Treatment | (MCT) | (%) | (%) | (g) | (g) | (g) | 5x) | pressure) |
| 1A | 5% | 5.00% | 90.00% | 10 | 10 | 180 | 5 | 800 |
| 1B | 5% | 10.00% | 85.00% | 10 | 20 | 170 | 5 | 800 |
| 1C | 5% | 15.00% | 80.00% | 10 | 30 | 160 | 5 | 800 |
| 2A | 5% | 5.00% | 90.00% | 10 | 10 | 180 | 5 | 300 |
| 2B | 5% | 5.00% | 90.00% | 10 | 10 | 180 | 5 | 1200 |
| 2C | 5% | 5.00% | 90.00% | 10 | 10 | 180 | 3 | 1500 |
| 3A = 5C-X1 | 5% | 2.50% | 92.50% | 10 | 5 | 185 | 5 | 800 |
| 3B | 5% | 3.75% | 91.25% | 10 | 7.5 | 182.5 | 5 | 800 |
| 3C | 5% | 3.00% | 92.00% | 10 | 6 | 184 | 5 | 800 |
| 4A | 5% | 5.00% | 90.00% | 10 | 10 | 180 | 5 | 800 |
| 4B | 5% | 5.00% | 90.00% | 10 | 10 | 180 | 5 | 800 |
| 4C | 5% | 6.00% | 89.00% | 10 | 12 | 178 | 5 | 800 |
| 4D | 5% | 8.50% | 86.50% | 10 | 17 | 173 | 5 | 800 |
| 4E | 5% | 8.50% | 86.50% | 10 | 17 | 173 | 5 | 800 |
| 5A | 5% | 2.83% | 92.17% | 10 | 5.667 | 184.33 | 5 | 800 |
| 5B | 5% | 5.67% | 89.33% | 10 | 11.334 | 178.66 | 5 | 800 |
| 5C | 5% | 8.50% | 90.00% | 10 | 17.001 | 180 | 5 | 800 |
| 6A = 5C-X1 | 5% | 2.50% | 92.50% | 10 | 5 | 185 | 5 | 800 |
| 6B = 4C-X2 | 5% | 6.00% | 89.00% | 10 | 12 | 178 | 5 | 800 |
| 6D = 4C-X2 | 5% | 6.00% | 89.00% | 10 | 12 | 178 | 5 | 800 |
| 6C = 4C-X2-500 mL | 5% | 6.00% | 89.00% | 10 | 12 | 178 | 5 | 800 |
| 7A | 2% | 18.00% | 80.00% | 4 | 36 | 160 | 5 | 800 |
| 7B | 5% | 10.00% | 85.00% | 10 | 20 | 170 | 5 | 800 |
| 7C | 5% | 5.00% | 90.00% | 10 | 10 | 180 | 5 | 800 |
| 7D | 5% | 10.00% | 85.00% | 10 | 20 | 170 | 5 | 800 |
| 7E | 5% | 15.00% | 80.00% | 10 | 30 | 160 | 5 | 800 |
| 8A | 5% | 10.00% | 85.00% | 10 | 20 | 170 | 5 | 800 |
| 8B | 5% | 10.00% | 85.00% | 10 | 20 | 170 | 5 | 800 |

### I. Results

### a) Ratio Emulsifier:Oil

### Tween 20+Cosurfactant Lecithin

| | | | | Turbidity | Turbidity |
|---|---|---|---|---|---|
| | | | Temp | Before | after 165x |
| Treatment | | | after cycles | dilution | dilution |
| description | Treatment | Emulsifier type | (° C.) | (NTU) | (NTU) |
| Emulsifier:oil | 1A-X2 | 65% Tween 20 + | 49.5 | 892 | 25.5 |
| (1:1) | | 35% lecithin | | | |
| Emulsifier:oil | 1B-X2 | 65% Tween 20 + | 49.4 | 605 | 12.6 |
| (2:1) | | 35% lecithin | | | |
| Emulsifier:oil | 7B-X1 | 65% Tween 20 + | 50 | 397 | 8.9 |
| (3:1) | | 35% lecithin | | | |

For the pre-solution: Tween 20 was solubilized in the organic phase, lecithin was solubilized in the aqueous phase.
The solution foamed.
The homogenizer was operating at 800 Bar for 5 passes.
1A with lowest emulsifier:oil ratio showed the highest turbidity (25.5 NTU).
1B and 7B showed a lower NTU, but tasted very bitter.

### b) Applied Pressure (Bar)

### Tween 20+Cosurfactant Lecithin

| | | | | | Temp | Turbidity | Turbidity |
|---|---|---|---|---|---|---|---|
| | | | | Bar | after | Before | after 165x |
| Treatment | | Emulsifier | | (homogenisation | cycles | dilution | dilution |
| description | Treatment | type | Passes | pressure) | (° C.) | (NTU) | (NTU) |
| Emulsifier:oil | 2A - X2 | 65% | 5 | 300 | 31.6 | >1000 | 119.0 |
| (1:1) | | Tween 20 + | | | | | |
| | | 35% | | | | | |
| | | lecithin | | | | | |
| Emulsifier:oil | 1A - X2 | 65% | 5 | 800 | 49.5 | 892 | 25.5 |
| (1:1) | | Tween 20 + | | | | | |
| | | 35% | | | | | |
| | | lecithin | | | | | |
| Emulsifier:oil | 2B - X2 | 65% | 5 | 1200 | 57.5 | 793 | 17.9 |
| (1:1) | | Tween 20 + | | | | | |
| | | 35% | | | | | |
| | | lecithin | | | | | |
| Emulsifier:oil | 2C - X2 | 65% | 3 | 1500 | 59 | 899 | 20.4 |
| (1:1) | | Tween 20 + | | | | | |
| | | 35% | | | | | |
| | | lecithin | | | | | |

For the pre-solution: Tween 20 was solubilized in the organic phase, lecithin was solubilized in the aqueous phase.
The homogenizer was operating between 300 and 1500 Bar. The temperature after cycles was measured between 31 and 59° C., respectively. The homogenizer was operating at 5 passes for bar pressure 300, 800 and 1200. For 1500 bar, 3 passes were conducted.
The solution foamed.
Treatment 2A with a pressure of 300 Bar showed the highest turbidity (119 NTU). Based on the pictures before and after dilution, the solution did not look optically clear.
Treatment 1A from a previous experiment with a pressure of 800 bar showed a turbidity of 25.5 NTU.
Treatment 2B with a pressure of 1200 Bar showed a slightly lower turbidity of 17.9 NTU.
Treatment 2C with a pressure of 1500 Bar and only 3 passes showed a turbidity of 20.4 NTU.
An increase in pressure (Bar) increased the temperature after homogenization. Therefore, a lower pressure is preferred.
When comparing treatment 2B (1200 bar, 5 cycles) and 2C (1500 bar, 3 cycles); a turbidity of approximately 18 and 20 NTU is observed, respectively. Furthermore, a temperature of 58° C. for 2B and 59° C. for 2C is measured. This indicates that when lowering the number of passes, the pressure may be increased in order to obtain the same turbidity and thereby clear solution. On the other hand, when a high pressure of 1500 is used, the temperature could increase up to 80° C. (when the number of cycles will be higher than 3). This is not preferred as (in a next step) vitamins will be added to the solution, which are sensitive to heat.
Conclusion: at 800 Bar and 5 passes, the solution showed the most optimal combination of a low NTU and a low temperature.

### c) Emulsifier Type and Concentration

### Tween 20+Cosurfactant Lecithin

| | | | | Turbidity | Turbidity |
|---|---|---|---|---|---|
| | | | Temperature | before | after |
| | Treatment | | after cycles | dilution | dilution |
| Treatment description | code | Emulsifier type | (° C.) | (NTU) | (NTU) |
| (Emulsifier + | 7A - X1 | 85% Tween 20 + 15% | 51 | 290 | 5.8 |
| cosurfactant):oil (3:1) | | lecithin | | | |
| (Emulsifiers + | 7B - X1 | 65% Tween 20 + 35% | 50 | 397 | 8.9 |
| cosurfactant):oil (3:1) | | lecithin | | | |
| (Emulsifier + | 7C - X1 | Tween20 + lecithin (1% of | 52 | 280 | 5.7 |
| cosurfactant):oil (3:1) | | total = 1%*300 g) | | | |

For the pre-emulsion: Tween 20 was solubilized in the organic phase, lecithin was solubilized in the aqueous phase.
The homogenizer was operating at 800 Bar for 5 passes.
Tween 20 makes the solution taste bitter; lecithin does not add bitterness to the solution. Lecithin tastes like soy, beany, grassy.
The solution foams after shaking.
7C showed the highest bitterness perception compared to 7A and 7B due to a high amount of Tween present in the solution. 7B had the lowest bitterness perception due to a lower amount of Tween used.
All treatments showed a turbidity<10 NTU. Based on the pictures before and after dilution, all treatments showed optically clear solutions.
Treatment 7B was chosen as the best treatment due to the lowest bitterness perception and an optically clear solution.

### Sucrose Monostearate—Solubilized in Cold Water

| | | | | Turbidity | Turbidity |
|---|---|---|---|---|---|
| | | | Temperature | before | after |
| | Treatment | | after cycles | dilution | dilution |
| Treatment description | code | Emulsifier type | (° C.) | (NTU) | (NTU) |
| Emulsifier:oil (from | 5A - X1 | Sucrose monostearate | 45 | >1000 | 209 |
| literature: 0.1:1) | | | | | |
| Emulsifier:oil (1:1) | 5B - X1 | Sucrose monostearate | N/A | N/A | N/A |
| Emulsifier:oil (0.5:1) | 5C - X1 | Sucrose monostearate | 48 | 575 | 51 |
| Emulsifier:oil (0.75:1) | 3B - X2 | sucrose mono stearate | 42 | 583 | 60 |

For the pre-solution: Sucrose monostearate was solubilized in the aqueous phase (cold water), and stored overnight at 4° C.
The homogenizer was operating at 800 Bar for 5 passes.
Emulsifier:oil (1:1) was way too viscous, therefore treatment 5B could not be homogenized.
All treatments tasted like water, no bitterness or off-flavor perceived. However, turbidity is too high.
The lowest turbidity of 51 NTU is shown for sample 5C-X1. Based on the pictures before and after dilution, all treatments did not show optically clear solutions.
Treatment 5C-X1 was chosen as the best treatment due to the lowest NTU.

### Sucrose Monostearate—Solubilized in Hot Water (60-70 C)

| | | | | Turbidity |
|---|---|---|---|---|
| | | | Temperature | after |
| Treatment | Treatment | Emulsifier | after cycles | dilution |
| description | code | type | (° C.) | (NTU) |
| Emulsifier:oil (from | 5C-X1 | Sucrose | 45 | 59 |
| literature: 0.1:1) | | monostearate | | |
| Emulsifier:oil (1:1) | 5B-X1 | Sucrose | 52.00 | 35 |
| | | monostearate | | |
| Emulsifier:oil (2:1) | 5D | Sucrose | 51 | 26 |
| | | monostearate | | |

For the pre-solution: dissolved sucrose monostearate in warm water (62 degrees) (recommended by the supplier, Sisterna B.V.). Leave for 1 hour to solubilize and cool down, add oil phase, homogenized straight after adding oil.
The homogenizer was operating at 800 Bar for 5 passes.
The emulsifier concentration could be increased up to Emulsifier:oil (2:1) ratio, due to heating of the sucrose monostearate in the pre-solution.
After homogenization the treatments were still low in viscosity, however, after a couple of days, the hot solubilized treatments all became very viscous (thickness of yoghurt or even thicker with increasing sucrose monostearate concentration). Therefore, future experiments will be conducted with the cold solubilized sucrose monostearate.

### Monoglyceride

N/A, because monoglyceride has a very low HLB and is therefore suitable for a water in oil emulsion. For this experiment, we are only using emulsifiers for an oil in water emulsion as they perform best.

### Tween 80+Cosurfactant Lecithin

| | | | | Turbidity | Turbidity |
|---|---|---|---|---|---|
| | | | Temperature | before | after |
| | Treatment | | after cycles | dilution | dilution |
| Treatment description | code | Emulsifier type | (° C.) | (NTU) | (NTU) |
| Emulsifier:oil (1:1) | 5A-X2 | 21.7% Tween 80 + 35% | 45 | 883 | 26.4 |
| | | lecithine | | | |
| Emulsifier:oil (2:1) | 5B-X2 | 21.7% Tween 80 + 35% | 51 | 850 | 20 |
| | | lecithine | | | |
| Emulsifier:oil (3:1) | 5C-X2 | 21.7% Tween 80 + 35% | 53 | 365 | 10 |
| | | lecithine | | | |

For the pre-solution: Tween 80 was solubilized in the organic phase, lecithin was solubilized in the aqueous phase.
The homogenizer was operating at 800 Bar for 5 passes.
65% Tween 80 divided by 3=21.7%. A lower percentage is added because Tween 80 is three times more concentrated than Tween 20.
All treatments tasted slightly bitter and foamed. However, bitterness perception is lower compared to Tween 20 treatments.
The lowest turbidity of 10 NTU is shown for sample 5C-X2.
Treatment 5C-X2 was chosen as the best treatment due to the lowest NTU.

### Q-Naturale

| | | | | Turbidity | Turbidity |
|---|---|---|---|---|---|
| | | | Temperature | before | after |
| | Treatment | | after cycles | dilution | dilution |
| Treatment description | code | Emulsifier type | (° C.) | (NTU) | (NTU) |
| Emulsifier:oil (Ratio | 4A-X1 | Q-naturale | 43 | >1000 | 188 |
| from Ingredion: 1:4) | | | | | |
| Emulsifier oil (1:1) | 4B-X1 | Q-naturale | 46.6 | >1000 | 163 |
| Emulsifier:oil (2:1) | 4C-X1 | Q-naturale | 48.6 | >1000 | 115 |
| Emulsifier:oil (3:1) | 4D-X1 | Q-naturale | 50 | >1000 | 117 |

For the pre-solution: Q-naturale was solubilized in the aqueous phase.
The homogenizer was operating at 800 Bar for 5 passes.
You can taste the Q-naturale (tastes and smells like a sour/rancid off-flavor) for treatment 4B, 4C and 4D with an increased potency, respectively.
The Q-naturale samples foamed, except for 4A.
The lowest turbidity was measured at 115 NTU, which does not result in an optically clear solution.

### Mix of the Best Performing Emulsifiers so Far (Sucrose Monostearate+Tween 80)

| | | | | Turbidity | Turbidity |
|---|---|---|---|---|---|
| | | | Temperature | 165x | 500x |
| Treatment | Treatment | | after cycles | dilution | dilution |
| description | code | Emulsifier type | (° C.) | (NTU) | (NTU) |
| Emulsifier:oil | 5C-X1 | Sucrose | 53 | 54 | 22 |
| (0.5:1) | | monostearate | | | |
| Emulsifier:oil | 5C-X2 | 21.7% Tween 80 + | 48 | 10 | 2.9 |
| (3:1) | | 35% lecithin | | | |
| Emulsifier:oil | 4A-X2 | 50% sucrose | 49 | 12 | 3.5 |
| (1:1) | | monostearate + 50% | | | |
| | | tween 80 | | | |
| Emulsifier:oil | 4B-X2 | 50% sucrose | 46 | 10 | 4.7 |
| (1.15:1) | | monostearate + 65% | | | |
| | | tween 80 | | | |
| Emulsifier:oil | 4C-X2 | 50% sucrose | 51 | 10.2 | 2.5 |
| (1.1:1) | | monostearate + 50% | | | |
| | | tween 80 + | | | |
| | | (1%*total lecithine) | | | |
| Emulsifier:oil | 4D-X2 | 29.4% sucrose | 48 | 23.6 | 10 |
| (1.7:1) | | monostearate + | | | |
| | | 29.4% tween 80 + | | | |
| | | 41.18% g lecithine | | | |
| Emulsifier:oil | 4E-X2 | 29.4% sucrose | 46 | 20 | 14 |
| (1.7:1) | | monostearate + | | | |
| | | 17.6% tween 80 + | | | |
| | | 52.9% lecithine | | | |

For the pre-solution: Tween 80 was solubilized in the organic phase, sucrose monostearate and lecithin were solubilized in the aqueous phase.
The homogenizer was operating at 800 Bar for 5 passes.
Sample 4A and 4C obtained the same turbidity value with a lower amount of Tween 80 used. Furthermore, sample 4A and 4C are the best performing treatments, as they have a slightly lower bitterness perception and produce a lower amount of foam compared to 5C-X2.
Sample 4D and 4E were produced to lower the % Tween 80 even more and compensate for this with an increase in % lecithin. However, decreasing the amount of Tween 80 to 30% or lower, did not result in a NTU<15.
Sample 4B was produced to check the absence of lecithin. This treatment showed a turbidity of 10 NTU, however, the bitterness perception was equal to 5C-X2.
Conclusion: Sample 4A and 4C are performing better than 5C-X2 because of their lower bitterness perception and a decrease in foam formation, whilst having the same turbidity.
   Dilution Range with an a) Bitter Tasting_Transparant, and b) Neutral Tasting_Cloudy Treatment

| | | | 165x | 330x | 660x | 500x | 1000x | 2000x |
|---|---|---|---|---|---|---|---|---|
| Treatment | Treatment | | dilution | dilution | dilution | dilution | dilution | dilution |
| description | code | Emulsifier type | (NTU) | (NTU) | (NTU) | (NTU) | (NTU) | (NTU) |
| Emulsifier:oil | 5C-X1 | Sucrose | 54 | 29.05 | 19 | 22 | 13.20 | 5.8 |
| (0.5:1) | | monostearate | | | | | | |
| Emulsifier:oil | 5C-X2 | 21.7% Tween 80 + | 10 | 4.28 | 2.3 | 2.9 | 1.6 | 0.2 |
| (3:1) | | 35% lecithin | | | | | | |
| Emulsifier:oil | 4C-X2 | 50% sucrose | 10.2 | 4.75 | 2.2 | 2.5 | 1.7 | 0.18 |
| (1.1:1) | | monostearate + | | | | | | |
| | | 50% tween 80 + | | | | | | |
| | | (1%*total | | | | | | |
| | | lecithine) | | | | | | |

165× dilution=would add up to 20 mg CBD in final drink when 20% CBD is used.
330× dilution=would add up to 20 mg when 40% CBD is used.
660× dilution=would add up to 20 mg when 80% CBD is used.
500× dilution=would add up to 10 mg when 20% CBD is used.
1000× dilution=would add up to 10 mg when 40% CBD is used.
2000× dilution=would add up to 10 mg when 80% CBD is used.
The samples did not contain the % CBD that is mentioned. In this experiment, only de dilution factors are tested.
The neutral_cloudy treatment 5C-X1 has no off-flavor and no foam formation. However, the turbidity is only accepted for 1000× and 2000× dilution (NTU<15).
For the bitter_transparant treatment 5C-X2, the transparency for all the dilution factors is accepted (NTU<15). However, the 330× and the 660× both have a very small bitterness and foam formation. The 1000× and 2000× bitterness is neglected, however the treatments still foam.
For the bitter_transparant treatment 4C-X2, the transparency for all the dilution factors is accepted (NTU<15). However, the 330× and 660× foam and have a light note of nutty due to the lecithin. 330× has a very small bitterness, but lower than 5C-X2. 1000× and 2000×: nutty and bitterness perception can be neglected, but the treatments still foam.
Conclusion: Treatment 5C-X1 has no off-flavor or foam formation. Only 1000× and 2000× dilution have transparency (NTU<15). Treatment 5C-X2+4C-X2 are transparent but all dilutions have foam formation, which makes these treatments less favorable compared to 5C-X1. Moreover, 4C-X2 foams less compared to 5C-X2.
   Experiment with 5%, 20 and 40% CBD in Oil, for an a) Bitter_Transparant, and b) Neutral_Cloudy Treatment

| | | | | Turbidity | Turbidity |
|---|---|---|---|---|---|
| | | | Temperature | 165x | 1000x |
| Treatment | Treatment | | after cycles | dilution | dilution |
| description | code | Emulsifier type | (° C.) | (NTU) | (NTU) |
| Emulsifier:oil | 6A-X2 = 5C- | Sucrose | | | |
| (0.5:1) with 20% | X1 | monostearate | 49 | 128 | n.a. |
| CBD from Becanex | | | | | |
| Emulsifier:oil | 6B-X2 = 4C- | 50% sucrose | 47 | 32 | 10 |
| (1.1:1) with 20% | X2 | monostearate + 50% | | | |
| CBD from Becanex | | tween 80 + (1%*total | | | |
| | | lecithin) | | | |
| Emulsifier:oil | 6D-X2 = 4C- | 50% sucrose | To be tested | | |
| (1.1:1) with 5% | X2 | monostearate + 50% | | | |
| CBD from Becanex | | tween 80 + (1%*total | | | |
| | | lecithin) | | | |
| Emulsifier:oil | 6C-X2= 4C- | 50% sucrose | 56 | 106 | n.a. |
| (1.1:1) with | X2 | monostearate + 50% | | | |
| 40% CBD | | tween 80 + (1%*total | | | |
| | | lecithin) | | | |

All treatments with CBD tasted extremely bitter. Especially treatment 4C-X2.
All 20% CBD treatments (MCT+CBD) showed an approximate 2-fold increase in NTU compared to the same treatments with just MCT oil. 5C-X1 without CBD showed a NTU of 54, while 5C-X1 with 20% CBD showed a NTU of 128 (after 165× dilution).
The 40% CBD treatment (6C) showed an approximate 10-fold increase in NTU compared to the same treatment with no CBD.

### Kolliphor RH40

The MCT oil phase and surfactant were heated to 50° C., mixed and stirred at 700 rpm for 5 min to form an isotropic mixture, the pre-emulsion. The pre-emulsion was then left to cool down to room temperature. As a next step, the pre-emulsion was mixed with the aqueous phase using an ultra turrax for 5 min at 12000 rpm and left for 1 hour prior to homogenization.

| | | | | Turbidity | Turbidity |
|---|---|---|---|---|---|
| | | | Temperature | before | after 165x |
| Treatment | Treatment | | after cycles | dilution | dilution |
| description | code | Emulsifier type | (° C.) | (NTU) | (NTU) |
| Emulsifier:oil (9:1) | 7A-X2 | Kolliphor RH 40 | 45 | 66 | 1.8 |
| Emulsifier:oil (1:1) | 7C-X2 | Kolliphor RH 40 | 48 | 929 | 20.1 |
| Emulsifier:oil (2:1) | 7D-X2 | Kolliphor RH 40 | 50 | 507 | 8.7 |
| Emulsifier:oil (3:1) | 7E-X2 | Kolliphor RH 40 | 51 | 242 | 4 |

For treatment 7A: After mixing the aqueous with the oil phase the solution became clearer when letting the mixture rest for +−10 minutes. This can be explained through a phenomenon called spontaneous emulsification. This treatment became clear without any homogenization; however, the bitterness perception was high due to a high ratio of emulsifier to oil used (9:1).
Treatment 7C showed a turbidity of 20 NTU and no bitterness was perceived.
Treatment 7D showed a turbidity of 9 NTU with a very low bitterness perception that could easily be masked by addition of flavors. Treatment 7E showed a lower turbidity (4 NTU) after 165× dilution. Nevertheless, the bitterness perception was higher compared to 7C and 7D.
Treatment 7D was chosen as the best treatment due to a low off-flavor formation and an NTU<15.

As a next step, 7D-X2 was tested with 5% and 20% CBD added to the oil phase.

| | | | | Turbidity | Turbidity |
|---|---|---|---|---|---|
| | | | Temperature | before | after 165x |
| Treatment | Treatment | | after cycles | dilution | dilution |
| description | code | Emulsifier type | (° C.) | (NTU) | (NTU) |
| Emulsifier:oil (2:1) | 8A-X2 | Kolliphor RH40 + | 45 | 447 | 5.9 |
| | | 5% CBD | | | |
| Emulsifier:oil (2:1) | 8B-X2 | Kolliphor RH40 + | 46 | 381 | 5.4 |
| | | 20% CBD | | | |

Treatment 8A and 8B both showed a turbidity of approximately 5 NTU.
Bitterness was perceived, but only due to addition of CBD.
Out of all emulsifiers, Kolliphor RH40 showed the most optimal conditions.

### OSA Starch

| | | | | Turbidity | |
|---|---|---|---|---|---|
| | | | Temperature | after 165x | |
| Treatment | Treatment | | after cycles | dilution | |
| description | code | Emulsifier type | (° C.) | (NTU) | Bitterness |
| Emulsifier:oil | 2A-X1 | Tween 80 + OSA starch | 49 | 20 | High |
| (1 × 2:1) + Becanex | | | | | |
| 20% CBD oil | | | | | |
| Emulsifier:oil | 2B-X1 | Tween 80 + OSA starch | 46 | 8.1 | High |
| (2 × 2:1) + Becanex | | | | | |
| 20% CBD oil | | | | | |
| Emulsifier:oil | | | | | High |
| (3 × 2:1) + Becanex | 2C-X1 | Tween 80 + OSA starch | N/A | N/A | |
| 20% CBD oil | | | | | |
| Emulsifier oil (3.5:1) | 2D-X1 | Tween 80 + OSA starch | 49 | 59 | High |
| (tween80 = 0.5, OSA | | | | | |
| starch = 3) + Becanex | | | | | |
| 20% CBD oil | | | | | |
| Emulsifier:oil (1:1) + | 3A-X1 | OSA starch | 46 | 361 | Medium |
| Becanex 20% CBD oil | | | | | |
| Emulsifier:oil (2:1) + | 3B-X1 | OSA starch | 47 | 99 | Medium |
| normal MCT oil | | | | | |
| with no CBD | | | | | |
| Emulsifier oil (3:1) + | 3C-X1 | OSA starch | 46 | 374 | Medium |
| Becanex 20% CBD oil | | | | | |
| Emulsifier:oil (5:1) + | 3D-X1 | OSA starch | 40 | 321 | Medium |
| Becanex 20% CBD oil | | | | | |

1 day in advance, solubilize OSA starch in cold water.
Treatment 2A and 2B showed a NTU of 20 and 8.1, respectively. However, treatment 2B separated in 2 layers (oil and aqueous) after homogenization. Treatment 2A did not foam, however, the bitterness is high.
Treatment 2C and 2D were not suitable because 2A showed a too high viscosity for the homogenizer and 2D separated in an oil and aqueous layer after homogenization.
One unanticipated finding was that treatments 3A to 3D, with only OSA starch as emulsifier, showed a masking effect/decrease on the CBD bitterness. However, the NTU of the treatments is too high.
Treatment 3B, with no CBD added, showed no bitterness or off-flavor from OSA starch itself. In addition, it does not foam.

### OSA Starch

| | | | | Turbidity |
|---|---|---|---|---|
| | | | Temperature | after 165x |
| Treatment | Treatment | | after cycles | dilution |
| description | code | Emulsifier type | (° C.) | (NTU) |
| Emulsifier:oil (3 × 2:1) + | X3-1D | OSA starch + Tween80 | 27 | 10.2 |
| Becanex 20% CBD oil | | (300 Bar) | | |
| Emulsifier:oil (3 × 2:1) + | X3-1D warm | OSA starch + Tween80 | 25 | 8.5 |
| Becanex 20% CBD oil | | (300 Bar) | | |
| Emulsifier:oil (3 × 2:1) + | X3-1E | OSA starch + Tween80 | 27 | 13.2 |
| Becanex 20% CBD oil | | (300 Bar) | | |

1 day in advance, solubilize OSA starch in cold water (high foam formation occurred), and stirred overnight. Tween 80 in oil phase. For treatment X3-1Dwarm, the OSA starch was first dispersed in water at 70 C then stirred overnight to enhance the hydration of the starch.
The homogenizer was operating at 300 Bar for 3 passes.
All treatments showed an acceptable turbidity>15 NTU.
However, all treatments showed a phase separation (layer of oil and aqueous).
The bitterness perception of all treatments was high.

### Some Further Text Protocols

### Lemon-Lime 6A-X2

Lemon-lime 6A-X2

| Concentrat- - before dilution | | | | Added after dilution | | |
|---|---|---|---|---|---|---|
| | | | g | | | g |
| Ingredients | Phase | % w/w | 1000 | Ingredients | % w/w | 330 |
| Sucrose monostearate | AQUEOUS | 2.50% | 25 | Kanegrade | 0.40% | |
| | PHASE | | | lemon juice | | |
| | | | | concentrate | | |
| 50% citric acid (g/L) | | 0.3% | 3 | modifier | 0.10% | |
| | | | | sweet | | |
| 30% potassium sorbate | | 0.15% | 1.5 | stevia | 0.00% | |
| (g/L) | | | | | | |
| Water (g/L) | | | 0 | givaudon lime | 0.03% | |
| | | | | flavouring | | |
| Omega 3 | | | 0 | | | |
| ALL-Q ® (CoQ10) | | | 0 | | | |
| resVida ® (Resveratrol) | | | 0 | | | |
| MCT oil + CBD (g/L) | OIL | 5% | 50 | | | |
| Vitamin D | PHASE | | 0 | | | |
| luteine + zeaxanthine | | | 0 | | | |
| Vitamin E | | | 0 | | | |

### Lemon-Lime 6B-X2

Lemon-lime 6B-X2

| Concentrat- - before dilution | | | | Added after dilution | | |
|---|---|---|---|---|---|---|
| | | | g | | | g |
| Ingredients | Phase | % w/w | 1000 | Ingredients | % w/w | 330 |
| Soy lecithin | AQUEOUS | 1.00% | 10 | Kanegrade | 0.40% | |
| | PHASE | | | lemon juice | | |
| | | | | concentrate | | |
| Sucrose monostearate | | 2.50% | 25 | modifier sweet | 0.10% | |
| 50% citric acid (g/L) | | 0.3% | 3 | stevia | 0.00% | |
| 30% potassium sorbate | | 0.15% | 1.5 | givaudon lime | 0.03% | |
| (g/L) | | | | | | |
| Water (g/L) | | | 0 | | | |
| Omega 3 | | | 0 | | | |
| ALL-Q ® (CoQ10) | | | 0 | | | |
| resVida ® (Resveratrol) | | | 0 | | | |
| MCT oil + CBD (g/L) | OIL | 5% | 50 | | | |
| Polysorbate 80 (g/L) | PHASE | 2.50% | 25 | | | |
| Vitamin D | | | 0 | | | |
| luteine + zeaxanthine | | | 0 | | | |
| Vitamin E | | | 0 | | | |

### Lemon-Lime 8B-X2

Lemon-lime 8B-X2

| Concentrat- - before dilution | | | | Added after dilution | | |
|---|---|---|---|---|---|---|
| | | | g | | | g |
| Ingredients | Phase | % w/w | 1000 | Ingredients | % w/w | 330 |
| Kolliphor RH40 | AQUEOUS | 10.00% | 100 | Kanegrade | 0.40% | |
| | PHASE | | | lemon juice | | |
| | | | | concentrate | | |
| 50% citric acid (g/L) | | 0.3% | 3 | modifier | 0.10% | |
| | | | | sweet | | |
| 30% potassium sorbate (g/L) | | 0.15% | 1.5 | stevia | 0.00% | |
| Water (g/L) | | | 0 | givaudon lime | 0.03% | |
| | | | | flavouring | | |
| Omega 3 | | | 0 | | | |
| ALL-Q ® (CoQ10) | | | 0 | | | |
| resVida ® (Resveratrol) | | | 0 | | | |
| MCT oil + CBD (g/L) | OIL | 5% | 50 | | | |
| Vitamin D | PHASE | | 0 | | | |
| luteine + zeaxanthine | | | 0 | | | |
| Vitamin E | | | 0 | | | |

### (OSA Starch—2-X1)

| Ingredients | Phase | % w/w | 1000 g |
|---|---|---|---|
| 30% OSA starch (g/L) + | AQUEOUS | 50 (15% OSA | 500 (150 g OSA |
| 70% water = (pre-emulsion) | PHASE | starch + 35% water) | starch and 350 g water) |
| 50% citric acid (g/L) | | 0.240 | 2.4 |
| 30% potassium sorbate (g/L) | | 0.132 | 1.32 |
| Water (g/L) | | 29.628 | 296.28 |
| Kanegrade lemon juice | | 0.40 | Added after dilution |
| concentrate | | | |
| modifier sweet | | 0.10 | Added after dilution |
| stevia | | 0.0040 | Added after dilution |
| givaudon lime flavouring | | 0.03 | Added after dilution |
| Omega 3 | | | |
| ALL-Q ® (CoQ10) | | | |
| res Vida ® (Resveratrol) | | | |
| MCT oil + CBD (g/L) | OIL PHASE | 5 | 50 |
| Polysorbate 80 (g/L) | | 15 | 150 |
| Vitamin D | | | |
| luteine + zeaxanthine | | | |
| Vitamin E | | | |

### d) Carrier Oils

### 5-MCT, Hosun, or Essential Oils

As carrier oils, MCT and Hosun were chosen. These are very neutral in taste and stable. Vegetable oils and flavor oils differ in the chain length of the fatty acids of the triglyceride. Vegetable oils are mostly composed of long-chain fatty acids (C18) and medium-chain fatty acids (C12-C14) while flavor oils mainly contain short-chain fatty acids (<C10). In general, short-chain triglycerides (essential oils) can form emulsions with droplet sizes as small as 10 nm, while with long-chain triglycerides, the smallest particle size is typically around 100 nm. Therefore, in addition to MCT and Hosun, certain treatments could contain addition of an essential oil (e.g., citrus oil).

### Addition of Essential Oils

| | | | | Turbidity | Turbidity |
|---|---|---|---|---|---|
| | | | Temperature | before | after |
| Treatment | Treatment | Emulsifier | after cycles | dilution | dilution |
| description | code | type | (° C.) | (NTU) | (NTU) |
| Essential | 10A-X1 | 65% Tween | 47 | 560 | 26.3 |
| oil:MCT (4:1) + | | 20 + 35% | | | |
| Emulsifier:oil | | lecithine | | | |
| (1:1) | | | | | |
| Essential | 10B-X1 | 65% Tween | 49 | 867 | 19.8 |
| oil:MCT (1:4) + | | 20 + 35% | | | |
| Emulsifier:oil | | lecithine | | | |
| (1:1) | | | | | |
| Essential | 10C-X1 | 65% Tween | 49 | 555 | 12.4 |
| oil:MCT (1:1) + | | 20 + 35% | | | |
| Emulsifier:oil | | lecithine | | | |
| (1:1) | | | | | |

For the pre-solution: Tween 20 was solubilized in the organic phase, lecithin was solubilized in the aqueous phase.
The homogenizer was operating at 800 Bar for 5 passes.
The treatments foamed. They tasted bitter and you can taste the lemon from the essential oil.
The turbidity of 10C-X1 was low (12.4 NTU), however the taste of the essential oil was too overpowering, making the addition of essential oils unsuitable for this application.

In embodiments, one or more of the following may apply:
A turbidity of 15 NTU or lower is preferred.
The homogenizer protocol is most optimal when operating at 800 Bar for 5 passes.
Sucrose monostearate performed best in terms of no bitterness perception and no foam formation. However, the solution showed an NTU>15 and became extremely viscous after a couple days of storage, making this treatment unsuitable for future experiments.
Treatments 5C-X2 (Tween 80+lecithin) and 4C-X2 (sucrose monostearate+tween 80+lecithin) showed an acceptable turbidity with an NTU of 10. However, both treatments showed a slightly bitter off-flavor and foam formation, which are undesirable characteristics.
When CBD was added, the bitterness perception increased. Furthermore, the turbidity increased by a 2-fold when 20% CBD MCT oil was used.
Treatment 7D-X2 (Kolliphor RH40) with an emulsifier:oil ratio of (2:1) showed a turbidity of 9 NTU whilst having a lower bitterness perception compared to 5C-X2 and 4C-X2. Moreover, the low bitterness of 7D-X2 could easily be masked by addition of flavors and sweeteners. When 20% CBD is added to the oil phase, the 165× dilution showed a NTU of 5, with 20 mg CBD in the final drink when 20% CBD is used.
The turbidity and bitterness perception of 7D-X2 are acceptable which makes this the best performing treatment so far.

The following may be possible in embodiments:
1. Using absolute ethanol as a cosurfactant, having an emulsifier:oil ratio of (2:1) and an oil:ethanol ratio of (2:1). This could decrease the turbidity of the solution.
2. Using emulsifier OSA.
3. Using addition of other ingredients, such as sweeteners, flavors, oil soluble vitamins, acidity regulators, and/or preservatives. (For example: citric acid, potassium sorbate, sucralose, vitamin A, D, K, and flavors).

In an example, the first mixture may comprise about 5% oil+CBD, wherein about 5-40 of the oil+CBD is CBD, about 0.5-30% emulsifiers, optional further additives (in embodiments less than about 5 wt %), and the remaining is water (up to 100 wt %).

FIGS. 1a-1b schematically depict some aspects of the invention.

FIG. 1a schematically depicts in embodiments I and II embodiments of a method for producing a liquid, wherein the method comprises a first stage for providing a first liquid comprising components, wherein the components comprise water, oil, an emulsifier, and a functional component, wherein the first stage comprises: (a) a first mixing stage, wherein the first mixing stage comprises: combining and mixing water, oil, an emulsifier, and the functional component, to provide a first mixture, wherein in the first mixing stage a mixer device is applied rotating at a speed of at least 5000 RPM; and wherein the functional component comprises Cannabidiol; and (b) a first homogenization stage to provide the first liquid, wherein the first homogenization stage comprises homogenizing in a homogenizer n times the first mixture, wherein in the first homogenization stage a homogenizer is applied at a pressure of at least 250 bar; wherein n is selected from the range of 1-10.

Here, in embodiment I the first mixing stage all components C are combined and mixed.

Here, in embodiment II the following may apply: (a) the components comprise a first emulsifier, wherein the first mixing stage comprises a first premixing stage comprising: (i) combining and mixing the Cannabidiol, a food-grade oil, the first emulsifier, and optionally a further first premixing stage mixture component, (ii) optionally subjecting the thus obtained first premixing stage mixture to a heating stage, wherein the thus obtained mixture is subjected to a temperature selected from the range of 20-60° C., especially wherein the thus obtained mixture is subjected to a temperature selected from the range of 30-60° C. during a time period of at least 15 minutes; (b) the components comprise a second emulsifier, wherein the first mixing stage comprises a second premixing stage comprising: combining and mixing the second emulsifier and water to provide a second premixing stage mixture; and (c) the first mixing stage comprises: combining and mixing the first premixing stage mixture and the second premixing stage mixture, to provide the first mixture.

Hence, a first mixture may be obtained as such due to combining and mixing the components during a first mixing stage, whereafter the first mixture is homogenized in the first homogenization stage, to provide a homogenized first mixture.

A first mixture may also be obtained by executing the first premixing stage and the second premixing stage, and combining and mixing the first premixing stage mixture and the second stage mixture to provide the first mixture in the first mixing stage, i.e. especially wherein the mixer device is applied rotating at a speed of at least 5000 RPM, whereafter the first mixture is homogenized in the first homogenization stage, to provide a homogenized first mixture.

Referring to FIG. 1b, two embodiments are schematically depicted. In the first embodiment, the second liquid is obtained and in the second embodiment a dried product is obtained. The latter may optionally be diluted to arrive at the second mixture/second liquid. FIG. 1b, embodiment I, schematically depicts an embodiment of the method comprising a second stage for providing a second liquid comprising the first liquid, wherein the second stage comprises: a second mixing stage, wherein the second mixing stage comprises combining and mixing the first liquid and an aqueous liquid, to provide the second mixture; especially wherein the aqueous liquid is water. FIG. 1b, embodiment II, schematically depicts an embodiment of the method comprising a spray drying stage, wherein the spray drying stage comprises: combining and mixing the first mixture with an additive comprising one or more of a water soluble protein and a water soluble carbohydrate, and spray drying the thus obtained mixture to provide a spray dried product. FIG. 1b, embodiment II, schematically also depicts an embodiment wherein the method comprises a third stage for providing a second liquid, wherein the third stage comprises: a third mixing stage, wherein the third mixing stage comprises combining and mixing the spray dried product and an aqueous liquid, to provide the second mixture; especially wherein the aqueous liquid is water.

Here below, the references (Refs) in the drawings are further elucidated:

| Ref. | Indication | Ref. | Indication |
|---|---|---|---|
| C | components | Aq | Water |
| M1 | First mixture | M2 | second mixture |
| MS1 | First mixing stage | MS2 | Second mixing stage |
| HM1 | First homogenization stage | L2 | Second liquid |
| L1 | First liquid | A | Additive |
| PM1 | First premixing stage mixture | DS | (spray) drying stage |
| PM2 | Second premixing stage mixture | P1 | (spray) dried product |
| | | MS3 | Third mixing stage |

The term “plurality” refers to two or more.

The terms “substantially” or “essentially” herein, and similar terms, will be understood by the person skilled in the art. The terms “substantially” or “essentially” may also include embodiments with “entirely”, “completely”, “all”, etc. Hence, in embodiments the adjective substantially or essentially may also be removed. Where applicable, the term “substantially” or the term “essentially” may also relate to 90% or higher, such as 95% or higher, especially 99% or higher, even more especially 99.5% or higher, including 100%. The term “comprise” also includes embodiments wherein the term “comprises” means “consists of”. The term “and/or” especially relates to one or more of the items mentioned before and after “and/or”. For instance, a phrase “item 1 and/or item 2” and similar phrases may relate to one or more of item 1 and item 2. The term “comprising” may in an embodiment refer to “consisting of” but may in another embodiment also refer to “containing at least the defined species and optionally one or more other species”. Furthermore, the terms first, second, third and the like in the description and in the claims, are used for distinguishing between similar elements and not necessarily for describing a sequential or chronological order. It is to be understood that the terms so used are interchangeable under appropriate circumstances and that the embodiments of the invention described herein are capable of operation in other sequences than described or illustrated herein.

The devices, apparatus, or systems may herein amongst others be described during operation. As will be clear to the person skilled in the art, the invention is not limited to methods of operation, or devices, apparatus, or systems in operation.

It should be noted that the above-mentioned embodiments illustrate rather than limit the invention, and that those skilled in the art will be able to design many alternative embodiments without departing from the scope of the appended claims.

In the claims, any reference signs placed between parentheses shall not be construed as limiting the claim.

Use of the verb “to comprise” and its conjugations does not exclude the presence of elements or steps other than those stated in a claim. Unless the context clearly requires otherwise, throughout the description and the claims, the words “comprise”, “comprising”, and the like are to be construed in an inclusive sense as opposed to an exclusive or exhaustive sense; that is to say, in the sense of “including, but not limited to”. The article “a” or “an” preceding an element does not exclude the presence of a plurality of such elements.

The invention may be implemented by means of hardware comprising several distinct elements, and by means of a suitably programmed computer. In a device claim, or an apparatus claim, or a system claim, enumerating several means, several of these means may be embodied by one and the same item of hardware. The mere fact that certain measures are recited in mutually different dependent claims does not indicate that a combination of these measures cannot be used to advantage. In yet a further aspect, the invention (thus) provides a software product, which, when running on a computer is capable of bringing about (one or more embodiments of) the method as described herein.

The invention also provides a control system that may control the device, apparatus, or system, or that may execute the herein described method or process. Yet further, the invention also provides a computer program product, when running on a computer which is functionally coupled to or comprised by the device, apparatus, or system, controls one or more controllable elements of such device, apparatus, or system.

The invention further applies to a device, apparatus, or system comprising one or more of the characterizing features described in the description and/or shown in the attached drawings. The invention further pertains to a method or process comprising one or more of the characterizing features described in the description and/or shown in the attached drawings.

The various aspects discussed in this patent can be combined in order to provide additional advantages. Further, the person skilled in the art will understand that embodiments can be combined, and that also more than two embodiments can be combined. Furthermore, some of the features can form the basis for one or more divisional applications.

1. A method for producing a liquid, wherein the method comprises a first stage for providing a first liquid comprising components, wherein the components comprise water, oil, an emulsifier, and a functional component, wherein the functional component comprises Cannabidiol, wherein the first stage comprises:
a first premixing stage, wherein the first premixing stage comprises: combining and mixing the Cannabidiol, oil, and a first emulsifier to provide a first premixing stage mixture;
a first mixing stage, wherein the first mixing stage comprises: combining and mixing water and the first premixing stage mixture to provide a first mixture, wherein in the first mixing stage a mixer device is applied rotating at a speed of at least 5000 RPM; and
a first homogenization stage to provide the first liquid, wherein the first homogenization stage comprises homogenizing in a homogenizer n times the first mixture, wherein in the first homogenization stage a homogenizer is applied at a pressure of at least 250 bar; wherein n is selected from the range of 1-10.

2. The method according to claim 1, wherein the components are selected such and wherein the first stage is executed such that the first liquid has a turbidity of not more than 1000 Nephelometric Turbidity units;
wherein the components are selected such and wherein the first stage is executed such that the first liquid has a turbidity of not more than 500 Nephelometric Turbidity units.

3. The method according to claim 1, wherein the first mixing stage comprises the first premixing stage comprising:
(i) combining and mixing the Cannabidiol, the oil, wherein the oil comprises a food-grade oil, the first emulsifier, and
(ii) subjecting the thus obtained first premixing stage mixture to a heating stage, wherein the thus obtained mixture is subjected to a temperature selected from the range of 20-60° C.,
wherein the thus obtained mixture is subjected to a temperature selected from the range of 30-60° C. during a time period of at least 15 minutes.

4. The method according to claim 1, wherein the first emulsifier is selected from the group of quillaia, Arabic gum, octenyl succinic anhydride modified waxy starch, a polysorbate, sucrose monostearate, a monoglyceride, soy lecithin, and Kolliphor RH40;
wherein the first emulsifier is selected from the group of a polysorbate and Kolliphor RH40;
wherein the first premixing stage mixture comprises the first emulsifier in a ratio of 1:2 relative to the food-grade oil up to ratio of 4:1; and
wherein the oil is selected from the group of Cannabis sativa oil, MCT oil, and sunflower oil.

5. The method according to claim 1, wherein the first premixing stage mixture comprises Cannabidiol in an amount of 9-30 weight %.

6. The method according to claim 1, wherein the first premixing stage mixture comprises ethanol in an amount of 5-50 weight %.

7. The method according to claim 1, wherein the components comprise a second emulsifier, wherein the first mixing stage comprises a second premixing stage comprising: combining and mixing the second emulsifier and water to provide a second premixing stage mixture; and wherein the first emulsifier and the second emulsifier are different or comprise different compositions.

8. The method according to claim 7, wherein:
(a) the second emulsifier is selected from the group of quillaia, Arabic gum, octenyl succinic anhydride modified waxy starch, a polysorbate, sucrose monostearate, a monoglyceride, soy lecithin, and Kolliphor RH40; wherein the second emulsifier is selected from the group of a sucrose monostearate and soy lecithin; and/or
(b) wherein the second premixing stage mixture comprises the second emulsifier in an amount of 1-5 weight %.

9. The method according to claim 7, wherein the first mixing stage comprises: combining and mixing the first premixing stage mixture and the second premixing stage mixture, to provide the first mixture,
wherein the first mixture comprises Cannabidiol in an amount of 0.5-6 weight %.

10. (canceled)

11. The method according to claim 1, wherein one or more of the following applies:
(a) in the first mixing stage a mixer device is applied rotating at a speed of at least 10.000 RPM; and wherein the mixer device comprises an ultra turrax device, and
(b) in the first homogenization stage a homogenizer is applied at a pressure of at least 500 bar;
wherein n is selected from the range of 3-10; wherein during each homogenization the elevated pressure is applied during a time period selected from the range of 0.1-20 seconds.

12. The method according to claim 1, wherein the components further comprise one or more of citric acid, ascorbic acid, and sodium benzoate.

13. The method according to claim 1, wherein the method comprises a second stage for providing a second liquid comprising the first liquid, wherein the second stage comprises:
a second mixing stage, wherein the second mixing stage comprises combining and mixing the first liquid and an aqueous liquid, to provide the second mixture; wherein the aqueous liquid is water;
wherein the first liquid is selected such and wherein the second stage is executed such that the second liquid has a turbidity of not more than 50 Nephelometric Turbidity units, wherein the first liquid is selected such and wherein the second stage is executed such that the second liquid has a turbidity of not more than 25 Nephelometric Turbidity units; and
wherein the second mixing stage comprises diluting the first liquid with the aqueous liquid in the order of 100-2000 times, to provide the second mixture.

14. The method according to claim 13, wherein one or more of the following applies:
(i) the second mixture comprises Cannabidiol in an amount of 0.001-0.02 weight %,
(ii) the second mixture comprises the oil in an amount of 0.003-0.2 weight %, and
(iii) the second mixture comprises the emulsifier in an amount of 0.0025-0.1 weight % relative to the second mixture, and further comprising:
a spray drying stage, wherein the spray drying stage comprises:
combining and mixing the first mixture with an additive comprising one or more of a water-soluble protein and a water-soluble carbohydrate, and
spray drying the thus obtained mixture to provide a spray dried product.

15. (canceled)

16. The method according to claim 14, wherein one or more of the following applies:
(a) the additive comprises one or more of maltodextrin, pea protein, and soy protein,
(b) the method comprises spray drying at a temperature selected from the range of 80-175° C., and
(c) the spray dried product has less than 15 weight % water, wherein the spray dried product has 5-8 weight % water,
wherein the method comprises a third stage for providing a second liquid, wherein the third stage comprises:
a third mixing stage, wherein the third mixing stage comprises combining and mixing the spray dried product and water to provide the second mixture.

17. (canceled)

18. Use of the liquid or a spray dried product obtainable with the method according to claim 1.

19. A product comprising the liquid or a spray dried product obtainable with the method according to claim 1.

20. The product of claim 19, wherein the product is selected from the group of a medicament and a functional food, wherein the product comprises Cannabidiol and is for use in a medical treatment.

21. The product according to claim 19, wherein the product comprises the first liquid or the second liquid, wherein:
(a) when the product comprises the first liquid:
the product has a turbidity of not more than 500 Nephelometric Turbidity units;
wherein the product comprises, relative to the total weight of the product:
Cannabidiol in an amount of 0.5-6 weight %;
the oil in an amount of 0.3-20 weight %;
the emulsifier in an amount of 0.25-30 weight %; and
wherein the product further comprises water, and optionally ethanol;
(b) when the product comprises the second liquid:
the product has a turbidity of not more than 25 Nephelometric Turbidity units; and
wherein the product comprises, relative to the total weight of the product:
Cannabidiol in an amount of 0.001-0.06 weight %;
the oil in an amount of 0.003-0.2 weight %;
the emulsifier in an amount of 0.0025-0.3 weight %; and
wherein the product further comprises water, and optionally ethanol.

22. The product according to claim 19, wherein the product comprises the spray dried product, wherein the product comprises, relative to the total weight of the product:
(a) Cannabidiol in an amount of 1-7 weight %;
(b) the oil in an amount of 0.5-25 weight %;
(c) the emulsifier in an amount of 0.5-40 weight %;
(d) the additive, comprising one or more of a water soluble protein and a water soluble carbohydrate, in an amount of 20-95 weight %, and
(e) less than 15 weight % water.

23. A method for producing a liquid, wherein the method comprises a first stage for providing a first liquid comprising components, wherein the components comprise water, oil, an emulsifier, and a functional component, wherein the functional component comprises Cannabidiol, wherein the first stage comprises:
a first premixing stage, wherein the first premixing stage comprises: combining and mixing the Cannabidiol, oil, and a first emulsifier to provide a first premixing stage mixture;
a first mixing stage, wherein the first mixing stage comprises: combining and mixing water and the first premixing stage mixture to provide a first mixture, wherein in the first mixing stage a mixer device is applied, wherein the mixer device is an ultra turrax device; and
a first homogenization stage to provide the first liquid, wherein the first homogenization stage comprises homogenizing in a homogenizer n times the first mixture, wherein in the first homogenization stage a homogenizer is applied at a pressure of at least 250 bar; wherein n is selected from the range of 1-10.
